(19) United States
(12) Patent Application Publication
(10) Pub. No.: US 2003/0004326 A1
(43) Pub. Date: Jan. 2, 2003

(54) Kin28 polynucleotides and polypeptides and uses thereof

(76) Inventor: Liping Wu, Belmont, MA (US)

Correspondence Address: J. PETER FASSE, Fish & Richardson P.C., 225 Franklin Street, Boston, MA, 02110-2804, US

(21) Appl. No.: 10/151,763

(22) Filed: May 20, 2002

Related U.S. Application Data
(60) Provisional application No. 60/291,855, filed on May 18, 2001.

(51) Int. Cl. C07H 21/02; C07H 21/04

(52) U.S. Cl. 536/23.1

(57) ABSTRACT

Disclosed are Aspergillus KIN28 genes and polypeptides and their use in identifying antifungal agents.

### CROSS-REFERENCE TO RELATED APPLICATION

This application claims the benefit of priority from U.S. Provisional Patent Application No. 60/291,855, filed on May 18, 2001, which is incorporated herein by reference in its entirety.

### FIELD OF THE INVENTION

The invention relates to fungal genes and polypeptides and their use in identifying antifungal agents.

### BACKGROUND OF THE INVENTION

Aspergillus is a ubiquitous filamentous fungus. It is commonly found in soil, plant debris, and indoor air. The genus Aspergillus includes over 185 species. Approximately 20 species have been reported as pathogenic agents in humans. Among these, Aspergillus fumigatus is the most commonly isolated species. Other pathogenic Aspergillus species include Aspergillus flavus, Aspergillus niger, Aspergillus clavatus, Aspergillus glaucus group, Aspergillus nidulans, Aspergillus oryzae, Aspergillus terreus, Aspergillus ustus, and Aspergillus versicolor.

Aspergillus species play roles in at least three different clinical settings in humans: (i) opportunistic infections; (ii) allergic states; and (iii) toxicoses. Immunosuppression is the major factor predisposing to development of opportunistic Aspergillus infections, generally referred to as aspergillosis. The three principal forms of aspergillosis are allergic bronchopulmonary aspergillosis, pulmonary aspergilloma, and invasive aspergillosis. The clinical manifestation and severity of disease depends upon the immunologic state of the patient. Lowered host resistance due to such factors as underlying debilitating disease, neutropenia chemotherapy, disruption of normal flora, and an inflammatory response due to the use of antimicrobial agents and steroids can predispose a patient to colonization, invasive disease, or both. Aspergillus species are frequently secondary opportunistic pathogens in patients with bronchiectasis, carcinoma, other mycoses, and tuberculosis. Among filamentous fungi, Aspergillus is one of the most commonly isolated in invasive infections.

Almost any organ or system in the human body may be involved in Aspergillus infection. Onychomycosis, sinusitis, cerebral aspergillosis, meningitis, endocarditis, myocarditis, pulmonary aspergillosis, osteomyelitis, otomycosis, endophthalmitis, cutaneous aspergillosis, hepatosplenic aspergillosis, as well as Aspergillus fungemia, and disseminated aspergillosis may develop. Nosocomial occurrence of aspergillosis resulting from the use of catheters and other devices is also common.

Aspergillus species may be local colonizers in previously developed lung cavities due to tuberculosis, sarcoidosis, bronchiectasis, pneumoconiosis, ankylosing spondylitis or neoplasms, presenting as a distinct clinical entity, called an aspergilloma. Aspergilloma may also occur in the kidneys.

Some Aspergillus antigens are fungal allergens and may initiate allergic bronchopulmonary aspergillosis, particularly in atopic hosts. Certain Aspergillus species produce various mycotoxins, some of which are carcinogenic. Among these mycotoxins, aflatoxin is well-characterized and may induce hepatocellular carcinoma. It is mostly produced by Aspergillus flavus and contaminates foodstuffs, such as peanuts.

Aspergillus species can also cause infections in animals. For example, in birds, respiratory infections may develop due to Aspergillus. Aspergillus may also induce mycotic abortion in cattle and sheep. Ingestion of high amounts of aflatoxin may induce a lethal effect in poultry fed with grain contaminated with the toxin.

There are some treatments for these fungal infections. For example, two drugs are commonly used in the treatment of invasive aspergillosis; however, neither is completely satisfactory. Amphotericin B is given intravenously and has a number of toxic side effects. Itraconazole, which can be given orally, is often prescribed imprudently, encouraging the emergence of resistant fungal strains (Dunn-Coleman and Prade, Nature Biotechnology, 1998, 16:5). Resistance is also developing to synthetic azoles (such as fluconazole and flucytosine), and the natural polyenes (such as amphotericin B) are limited in use by their toxicity.

Fungicide resistance generally develops when a fungal cell or fungal population that originally was sensitive to a fungicide becomes less sensitive by heritable changes after a period of exposure to the fungicide. In certain applications, such as agriculture, it is possible to combat resistance through alteration of fungicides or by the use of fungicide mixtures. To prevent or delay the build up of a resistant pathogen population, different agents that are effective against a particular disease must be available. One way of increasing the number of available agents is to search for new site-specific inhibitors.

Consequently, antifungal drug discovery efforts have been directed at components of the fungal cell or its metabolism that are unique to fungi, and hence might be used as therapeutic targets of new agents that act on the fungal pathogen without undue toxicity to host cells. Such potential targets include enzymes critical to fungal cell wall assembly (U.S. Pat. No. 5,194,600) as well as topoisomerases (enzymes required for replication of fungal DNA). Two semisynthetic antifungal agents such as the echinocandins and the related pneumocandins are in late stage clinical trials. Both are cyclic lipopeptides produced by fungi that non-competitively inhibit β(1,3)-glucan synthase and thus interfere with the biosynthesis of the fungal cell wall. These clinical candidates are generally more water-soluble, and have improved pharmacokinetics and broader antifungal spectra than their natural parent compounds.

Because a single approach may not be completely effective against all fungal pathogens, and because of the possibility of developed resistance to previously effective antifungal compounds, there remains a need for new antifungal agents with novel mechanisms of action and improved or different activity profiles. There is also a need for agents which are active against fungi but are not toxic to mammalian cells, as toxicity to mammalian cells can lead to a low therapeutic index and undesirable side effects in the host (e.g., patient). An important aspect of meeting this need is the selection of an appropriate component of fungal structure or metabolism as a therapeutic target.

Even after a particular intracellular target is selected, the means by which new antifungal agents are identified pose certain challenges. Despite the increased use of rational drug design, a preferred method continues to be the mass screening of compound “libraries” for active agents by exposing cultures of fungal pathogens to the test compounds and assaying for inhibition of growth. In testing thousands or tens of thousands of compounds, however, a correspondingly large number of fungal cultures must be grown over time periods which are relatively long compared to most bacterial culture times. Moreover, a compound which is found to inhibit fungal growth in culture may be acting not on the desired target but on a different, less unique fungal component, with the result that the compound may act against host cells as well and thereby produce unacceptable side effects. Consequently, there is a need for an assay or screening method which more specifically identifies those agents that are active against a certain intracellular target. Additionally, there is a need for assay methods having greater throughput, that is, assay methods which reduce the time and materials needed to test each compound of interest.

### SUMMARY OF THE INVENTION

The invention is based on the discovery of KIN28 genes in two species of Aspergillus, Aspergillus nidulans and Aspergillus fumigatus. The genes appear to be orthologs of the Saccharomyces cerevisiae essential gene, KIN28. Essential genes are those which are required for the growth (such as metabolism, division, or reproduction) and/or survival of an organism. In other words, if the expression or activity of these genes or their products is inhibited, cell growth will be inhibited and/or the cell will die. Thus, essential genes can be used as screening targets to identify therapeutic agents, e.g., antifungal agents. These therapeutic agents can reduce or prevent growth, or decrease pathogenicity or virulence. It is often preferable that these therapeutic agents kill the organism.

In Saccharomyces cerevisiae, the Kin28 protein is a component of the transcription factor IIK (TFIIK) subcomplex of the transcription factor IIH (TFIIH) complex. Kin28, as part of the TFIIH complex, is responsible for the phosphorylation of the C-terminal repeat domain (CTD) of subunit 1 of RNA polymerase II. Upon phosphorylation of its CTD, RNA polymerase II initiates the transcription of genes containing RNA polymerase II promoters. This Kin28-mediated phosphorylation event is required for the effective initiation of transcription and/or the capping of RNA polymerase II transcripts. The important general role of Kin28 in transcription is consistent with its being an essential gene. Kin28 therefore constitutes a useful target for screening for antifungal agents.

Kin28 is a cyclin-dependent, serine/threonine protein kinase. Kin28 physically associates with its regulatory subunit, the cyclin Ccl1, in the context of the general transcription factor complex TFIIH. This pair of cyclin-dependent kinase (CDK) and cyclin, Kin28 and Ccl1, is essential for the initiation of RNA polymerase II transcription. Specifically, TFIIH complexes lacking the Ccl1 cyclin component lack CTD kinase activity.

As described herein, Aspergillus Kin28 nucleic acids and polypeptides can be used to identify therapeutic agents, e.g., antifungal agents. Activities of Aspergillus Kin28 polypeptides that can be effectively targeted by therapeutic agents include the ability to: (1) associate with a cyclin, e.g., Ccl1; (2) associate with a component of the TFIIH complex; (3) mediate serine/threonine kinase activity; (4) phosphorylate the CTD of RNA polymerase II; (5) promote RNA polymerase II-mediated transcription; (6) promote capping of an RNA polymerase II transcript; (7) promote the recruitment of a capping enzyme to the CTD of RNA polymerase II, e.g., a phosphorylated CTD; (8) have its kinase activity activated by phophorylation by a cyclin-dependent kinase-activating kinase (CAK); (9) regulate cell cycle progression; and (10) regulate DNA repair mechanisms.

Aspergillus nidulans KIN28 nucleotide sequences are depicted in FIGS. 1A-1B as SEQ ID NO: 1 (genomic sequence) and SEQ ID NO: 2 (partial cDNA sequence), with the amino acid sequence depicted in FIG. 3 as SEQ ID NO: 6. Aspergillus fumigatus KIN28 nucleotide sequences are depicted in FIGS. 2A-2B as SEQ ID NO: 3 (genomic sequence) and SEQ ID NO: 4 (cDNA sequence), with the amino acid sequence depicted in FIG. 3 as SEQ ID NO: 7. The open reading frame of Aspergillus fumigatus KIN28 (SEQ ID NO: 5) extends from the initiation codon to the nucleotide immediately before the termination codon of SEQ ID NO: 4. Thus, the present invention relates to novel Aspergillus Kin28 polypeptides and to KIN28 nucleotide sequences encoding the same. The present invention also relates to the use of the novel nucleic acid and amino acid sequences in the diagnosis and treatment of disease. The present invention further relates to the use of the novel nucleic acid and amino acid sequences to evaluate and/or to screen for agents that can modulate (increase or decrease) Kin28 activity, e.g., the ability to associate with a cyclin, e.g., Ccl1, the ability to promote RNA polymerase II-mediated transcription, and/or the ability to mediate serine/threonine kinase activity, e.g., the ability to phosphorylate the CTD of RNA polymerase II. In addition, the present invention relates to genetically engineered host cells that include or express the novel nucleic acid and amino acid sequences to evaluate and/or to screen for agents that can modulate Kin28 activity.

The Aspergillus Kin28 polypeptides of the present invention are obtainable from Aspergillus species, e.g., Aspergillus nidulans and Aspergillus fumigatus.

The Kin28 polypeptides of the present invention may be the same as the naturally occurring form or a variant, fragment, or derivative thereof. In addition, Kin28 can be an isolated Kin28 or a purified Kin28. The Kin28 can be obtained from or produced by any suitable source, whether natural or not, or it may be synthetic, semi-synthetic, or recombinant.

The KIN28 genes of the invention appear to be orthologs of a Saccharomyces cerevisiae gene that is essential for survival of that organism. Accordingly, the KIN28 nucleic acid sequences of the invention, and the Kin28 polypeptides of the invention, are useful targets for identifying compounds that are fungal inhibitors, e.g., inhibitors of Aspergillus, e.g., Aspergillus nidulans or Aspergillus fumigatus. Such inhibitors attenuate fungal growth by inhibiting an activity of the Kin28 polypeptide, e.g., the ability to associate with a cyclin, e.g., Ccl1, the ability to promote RNA polymerase II-mediated transcription, and/or the ability to mediate serine/threonine kinase activity, e.g., the ability to phosphorylate the CTD of RNA polymerase II, or by inhibiting transcription or translation of a naturally occurring KIN28 nucleic acid.

In one aspect, the invention features an isolated nucleic acid molecule selected from the group consisting of: (a) a nucleic acid molecule that encodes a polypeptide containing the amino acid sequence of SEQ ID NO: 6 or SEQ ID NO: 7; (b) a nucleic acid molecule that encodes a polypeptide containing at least 20 contiguous amino acids of SEQ ID NO: 6 or SEQ ID NO: 7; and (c) a nucleic acid molecule that encodes a variant of a polypeptide containing the amino acid sequence of SEQ ID NO: 6 or SEQ ID NO: 7, wherein the nucleic acid molecule hybridizes under stringent conditions to a nucleic acid molecule consisting of the nucleotide sequence of SEQ ID NO: 1, SEQ ID NO: 2, SEQ ID NO: 3, SEQ ID NO: 4, or SEQ ID NO: 5, or a complement of SEQ ID NO: 1, SEQ ID NO: 2, SEQ ID NO: 3, SEQ ID NO: 4, or SEQ ID NO: 5.

The isolated nucleic acid molecules described herein can be used in a method for identifying a an antifungal agent for the treatment of a fungal infection, the method including: (a) obtaining a first sample of cells and a second sample of cells, the first and second samples of cells being capable of expressing the nucleic acid molecule in the presence of a test compound; (b) contacting the first sample of cells with a test compound; and (c) comparing the growth of the first sample of cells with the growth of the second sample of cells; wherein growth of the first sample of cells slower than the growth of the second sample of cells indicates the test compound is an antifungal agent. The first and second samples of cells can contain fungal cells such as Aspergillus cells (e.g., Aspergillus nidulans or Aspergillus fumigatus).

The nucleic acid molecules described herein can also be used in a method for identifying a candidate compound for treating a fungal infection, the method including: (a) measuring the activity of a KIN28 gene comprising the sequence of SEQ ID NO: 1 or SEQ ID NO: 3 in a cell in the presence of a test compound; and (b) comparing the activity measured in step (a) to a level of activity of the KIN28 gene in a cell in the absence of the test compound; wherein a level of activity of the KIN28 gene measured in the presence of the test compound lower than the level of activity of the KIN28 gene measured in the absence of the test compound indicates that the test compound is a candidate compound for treating a fungal infection.

The nucleic acid molecules described herein can also be used in a method for identifying an agent that can affect KIN28 nucleic acid molecule expression, the method including: contacting an agent with the nucleic acid molecule; and measuring the expression of the nucleic acid molecule, where a difference between a) expression in the absence of the agent and b) expression in the presence of the agent is indicative that the agent can affect KIN28 expression.

The invention also features an isolated nucleic acid molecule selected from the group consisting of: (a) a nucleic acid molecule containing the nucleotide sequence of SEQ ID NO: 1, SEQ ID NO: 2, SEQ ID NO: 3, SEQ ID NO: 4, or SEQ ID NO: 5; (b) a nucleic acid molecule containing the nucleotide sequence of SEQ ID NO: 1, SEQ ID NO: 2, SEQ ID NO: 3, SEQ ID NO: 4, or SEQ ID NO: 5, wherein the thymines are replaced with uracils; (c) a nucleic acid molecule that is complementary to (a) or (b); and (d) fragments of (a), (b), or (c) that contain at least 50 contiguous nucleotides of SEQ ID NO: 1, SEQ ID NO: 2, SEQ ID NO: 3, SEQ ID NO: 4, or SEQ ID NO: 5 or a complement of SEQ ID NO: 1, SEQ ID NO: 2, SEQ ID NO: 3, SEQ ID NO: 4, or SEQ ID NO: 5.

Further, the invention features an isolated nucleic acid molecule selected from the group consisting of: (a) a nucleic acid molecule containing a nucleotide sequence which is at least about 60% identical, e.g., at least about 75%, 85%, 90%, 95%, 98%, or 100% identical to the nucleotide sequence of SEQ ID NO: 1, SEQ ID NO: 2, SEQ ID NO: 3, SEQ ID NO: 4, or SEQ ID NO: 5 or a complement thereof, wherein the percent identity is calculated using the GAP program in the GCG software package, using a gap weight of 5.000 and a length weight of 0.100; (b) a nucleic acid molecule containing a nucleotide sequence that hybridizes to a nucleic acid molecule consisting of the nucleotide sequence of SEQ ID NO: 1, SEQ ID NO: 2, SEQ ID NO: 3, SEQ ID NO: 4, or SEQ ID NO: 5 under stringent conditions, or a complement thereof; and (c) a nucleic acid molecule containing a nucleotide sequence that hybridizes under stringent conditions to a nucleic acid molecule consisting of the nucleotide sequence of the cDNA insert of a plasmid deposited with the ATCC as Accession Number______, the cDNA insert of a plasmid deposited with the ATCC as Accession Number______,or a complement thereof. A nucleic acid described herein can further include a vector nucleic acid sequence. A nucleic acid described herein can further include a nucleic acid sequence encoding a heterologous polypeptide.

Also included in the invention is a host cell that contains a nucleic acid molecule described herein. The host cell can be a mammalian host cell or a non-mammalian host cell.

In another aspect, the invention features an isolated polypeptide selected from the group consisting of: (a) a polypeptide containing the amino acid sequence of SEQ ID NO: 6 or SEQ ID NO: 7; (b) a polypeptide described herein having a sequence of at least 20 contiguous amino acids of SEQ ID NO: 6 or SEQ ID NO: 7; (c) a variant of a polypeptide containing the amino acid sequence of SEQ ID NO: 6, SEQ ID NO: 7, an amino acid sequence encoded by the cDNA insert of the plasmid deposited with ATCC as Accession Number______, or an amino acid sequence encoded by the cDNA insert of the plasmid deposited with ATCC as Accession Number______, wherein the polypeptide is encoded by a nucleic acid molecule that hybridizes under stringent conditions to the complement of a nucleic acid molecule consisting of the nucleotide sequence of SEQ ID NO: 1, SEQ ID NO: 2, SEQ ID NO: 3, SEQ ID NO: 4, or SEQ ID NO: 5; (d) a polypeptide encoded by a nucleic acid molecule containing a nucleotide sequence that is at least 60%, e.g., at least 75%, 85%, 90%, 95%, 98%, or 100% identical to SEQ ID NO: 1, SEQ ID NO: 2, SEQ ID NO: 3, SEQ ID NO: 4, or SEQ ID NO: 5, wherein the percent identity is calculated using the GAP program in the GCG software package, using a gap weight of 5.000 and a length weight of 0.100; and (e) a polypeptide comprising SEQ ID NO: 6 with up to 10 conservative amino acid substitutions, or SEQ ID NO: 7 with up to 10 conservative amino acid substitutions, wherein the polypeptide has KIN28 activity, e.g., the ability to associate with a cyclin, e.g., Ccl1, the ability to promote RNA polymerase II-mediated transcription, and/or the ability to mediate serine/threonine kinase activity, e.g., the ability to phosphorylate the CTD of RNA polymerase II. A polypeptide described herein can further contain a heterologous amino acid sequence.

Also included in the invention is an antibody that selectively binds to a polypeptide described herein.

In an embodiment, a polypeptide described herein can be used in a method for identifying an agent that can affect Kin28 polypeptide activity, the method including contacting an agent with the polypeptide; and measuring the activity of the polypeptide; where a difference between a) activity of the polypeptide in the absence of the agent and b) activity of the polypeptide in the presence of the agent is indicative that the agent can affect Kin28 polypeptide activity.

In another aspect, the invention features a method for producing a polypeptide, the method including culturing a host cell described herein under conditions in which the nucleic acid molecule is expressed, wherein the polypeptide is selected from the group consisting of: (a) a polypeptide comprising the amino acid sequence of SEQ ID NO: 6 or SEQ ID NO: 7; (b) a polypeptide described herein having a sequence of at least 20 contiguous amino acids of SEQ ID NO: 6 or SEQ ID NO: 7; (c) a variant of a polypeptide containing the amino acid sequence of SEQ ID NO: 6, SEQ ID NO: 7, an amino acid sequence encoded by the cDNA insert of the plasmid deposited with ATCC as Accession Number______, or an amino acid sequence encoded by the cDNA insert of the plasmid deposited with ATCC as Accession Number______, wherein the polypeptide is encoded by a nucleic acid molecule that hybridizes under stringent conditions to the complement of a nucleic acid molecule consisting of the nucleotide sequence of SEQ ID NO: 1, SEQ ID NO: 2, SEQ ID NO: 3, SEQ ID NO: 4, or SEQ ID NO: 5; (d) a polypeptide encoded by a nucleic acid molecule containing a nucleotide sequence that is at least 60%, e.g., at least 75%, 85%, 90%, 95%, 98%, or 100% identical to SEQ ID NO: 1, SEQ ID NO: 2, SEQ ID NO: 3, SEQ ID NO: 4, or SEQ ID NO: 5, wherein the percent identity is calculated using the GAP program in the GCG software package, using a gap weight of 5.000 and a length weight of 0.100; and (e) a polypeptide comprising SEQ ID NO: 6 with up to 10 conservative amino acid substitutions, or SEQ ID NO: 7 with up to 10 conservative amino acid substitutions, wherein the polypeptide has KIN28 activity, e.g., the ability to associate with a cyclin, e.g., Ccl1, the ability to promote RNA polymerase II-mediated transcription, and/or the ability to mediate serine/threonine kinase activity, e.g., the ability to phosphorylate the CTD of RNA polymerase II.

Since the KIN28 genes are orthologs of a gene essential for survival in Saccharomyces cerevisiae, nucleic acids encoding Kin28 can be used to identify antifungal agents.

Also included in the invention is a method for identifying a candidate antifungal agent, the method including: (a) obtaining a first cell and a second cell, the first and second cells being capable of expressing a KIN28 nucleic acid molecule; (b) contacting the first cell with a test compound; (c) determining the level of expression of KIN28 in the first and second cells; and (d) comparing the level of expression in the first cell with the second cell; wherein expression of KIN28 in the first cell less than expression of KIN28 in the second cell indicates that the test compound is a candidate antifungal agent; and wherein the KIN28 nucleic acid molecule encodes a polypeptide containing the amino acid sequence of SEQ ID NO: 6 or SEQ ID NO: 7 or a naturally occurring allelic variant of a polypeptide containing the amino acid sequence of SEQ ID NO: 6 or SEQ ID NO: 7, and wherein the KIN28 nucleic acid molecule hybridizes to a second nucleic acid molecule under stringent conditions, the second nucleic acid molecule consisting of the nucleotide sequence of SEQ ID NO: 1, SEQ ID NO: 2, SEQ ID NO: 3, SEQ ID NO: 4, or SEQ ID NO: 5 or the complement of SEQ ID NO: 1, SEQ ID NO: 2, SEQ ID NO: 3, SEQ ID NO: 4, or SEQ ID NO: 5. The level of expression can be measured by measuring the amount of KIN28 mRNA in the cell. Alternatively, the level of expression can be measured by measuring the amount of protein encoded by KIN28.

The test compound as described herein can be small organic or inorganic molecules. Alternatively, the test compound can be a test polypeptide (e.g., a polypeptide having a random or predetermined amino acid sequence; or a naturally-occurring or synthetic polypeptide) or a nucleic acid, such as a DNA or RNA molecule. The test compound can be naturally-occurring compound or it can be synthetically produced. Synthetic libraries, chemical libraries, and the like can be screened to identify compounds that bind to Kin28. The test compound can be, without limitation, a polypeptide, ribonucleic acid, a small organic molecule, a small inorganic molecule, a peptidomimetic, a polysaccharide,a deoxyribonucleic acid, an antisense oligonucleotide, or a ribozyme.

Another suitable method for identifying antifungal compounds involves screening for small molecules that specifically bind to Kin28. A variety of suitable binding assays are known in the art as described, for example, in U.S. Pat. Nos. 5,585,277 and 5,679,582, incorporated herein by reference. For example, in various conventional assays, test compounds can be assayed for their ability to bind to a polypeptide by measuring the ability of the small molecule to stabilize the polypeptide in its folded, rather than unfolded, state. More specifically, one can measure the degree of protection against unfolding that is afforded by the test compound. Test compounds that bind to a Kin28 with high affinity cause, for example, a significant shift in the temperature at which the polypeptide is denatured. Test compounds that stabilize the polypeptide in a folded state can be further tested for antifungal activity in a standard susceptibility assay.

In a variation of the above method, the invention features a method for identifying a candidate compound useful for treating a fungal infection, where the method entails (a) measuring the activity of KIN28 gene comprising the sequence of SEQ ID NO: 1 or SEQ ID NO: 3 in a cell in the presence of a test compound; and (b) comparing the activity measured in step (a) to a level of activity of the KIN28 gene in a cell in the absence of the test compound; wherein a level of activity of the KIN28 gene measured in the presence of the test compound lower than the level of activity of the KIN28 gene measured in the absence of the test compound indicates that the test compound is a candidate compound for treating a fungal infection.

In yet another embodiment, the invention features a method for identifying a candidate compound that may be useful for treating a fungal infection, wherein the method entails (a) contacting a variant, homolog, or ortholog of a Kin28 polypeptide with a test compound; (b) detecting binding of the test compound to the variant, homolog, or ortholog of Kin28; and (c) selecting as a candidate compound one that binds to the variant, homolog, or ortholog of Kin28, wherein Kin28 is encoded by a gene having the sequence of SEQ ID NO: 1 or SEQ ID NO: 3. Optionally, the method can also include (d) determining whether a candidate compound that binds to the variant, homolog, or ortholog of Kin28 inhibits growth of fungi, e.g., Aspergillus, relative to growth of fungi cultured in the absence of the candidate, where inhibition of growth indicates that the candidate compound is an antifungal agent. The variant, homolog, or ortholog can be derived from a non-pathogenic or pathogenic fungus.

The Kin28 polypeptides can be used in assays to identify test compounds that bind to the polypeptides. Test compounds that bind to Kin28 polypeptides then can be tested, in conventional assays, for their ability to inhibit fungal growth. Test compounds that bind to Kin28 polypeptides are candidate antifungal agents, in contrast to compounds that do not bind to Kin28 polypeptides. As described herein, any of a variety of art-known methods can be used to assay for binding of test compounds to Kin28 polypeptides. If desired, the test compound can be immobilized on a substrate, and binding of the test compound to Kin28 is detected as immobilization of Kin28 on the immobilized test compound. Immobilization of Kin28 on the test compound can be detected in an immunoassay with an antibody that specifically binds to Kin28.

Also included in the invention is a method for identifying a candidate antifungal agent useful for treating a fungal infection by: (a) contacting a Kin28 polypeptide encoded by a KIN28 nucleic acid molecule described herein with a test compound; and (b) detecting binding of the test compound to the polypeptide, wherein a test compound that binds to the Kin28 polypeptide indicates that the test compound is a candidate compound for treating a fungal infection. The method can further include determining whether the candidate compound that binds to the Kin28 polypeptide inhibits growth of fungi, relative to growth of fungi grown in the absence of the test compound, wherein inhibition of growth indicates that the candidate compound is an antifungal agent.

In one example, the test compound is immobilized on a substrate, and binding of the test compound to the Kin28 polypeptide is detected as immobilization of the Kin28 polypeptide on the immobilized test compound. Immobilization of the Kin28 polypeptide on the test compound can be detected in an immunoassay with an antibody that specifically binds to the Kin28 polypeptide.

In one example, the test compound is selected from the group consisting of a polypeptide, a ribonucleic acid, a small inorganic or organic molecule, a deoxyribonucleic acid, an antisense oligonucleotide, a peptidomimetic, a polysaccharide, and a ribozyme.

In another example, the Kin28 polypeptide is provided as a first fusion protein containing Kin28 polypeptide fused to (i) a transcription activation domain of a transcription factor or (ii) a DNA-binding domain of a transcription factor; and the test compound is a polypeptide that is provided as a second fusion protein containing the test compound fused to (i) a transcription activation domain of a transcription factor or (ii) a DNA-binding domain of a transcription factor, to interact with the first fusion protein; and binding of the test compound to the Kin28 polypeptide is detected as reconstitution of a transcription factor.

In another aspect, the invention features a method of treating a fungal infection in a subject, by administering to the subject an effective amount of an antifungal agent identified using any one of the methods described herein. In one example, the compound is selected from the group consisting of a polypeptide, ribonucleic acid, small inorganic or organic molecule, peptidomimetic, polysaccharide, and deoxyribonucleic acid. In another example, the compound is an antisense oligonucleotide. In another example, the compound is a ribozyme. The subject can be an animal or a person. Animals to be treated include mammals, such as dogs, cats, cows, pigs, sheep, and horses, as well as birds such as poultry.

In another aspect, the invention features a pharmaceutical formulation for the treatment of a fungal infection, the formulation containing an antifungal agent identified by a method described herein and a pharmaceutically acceptable excipient.

In another aspect, the invention features a method for treating an organism having a fungal infection, the method including administering to the organism a therapeutically effective amount of the pharmaceutical formulation described herein. The organism can be, for example, a human.

The invention also includes a method of treating an antifungal infection in an organism by administering to the organism a therapeutically effective amount of an antibody, e.g., a monoclonal antibody, described herein.

Also included in the invention is a pharmaceutical formulation for the treatment of a fungal infection in an organism, the formulation containing a ribozyme described herein and a pharmaceutically acceptable excipient.

Also included in the invention is a pharmaceutical formulation for the treatment of a fungal infection in an organism, the formulation containing an antisense nucleic acid described herein and a pharmaceutically acceptable excipient.

In another aspect, the invention features a method for identifying a candidate compound for treating a fungal infection, the method including: (a) contacting a KIN28 nucleic acid molecule with a test compound; and (b) detecting binding of the test compound to the KIN28 nucleic acid molecule, wherein a compound that binds to the KIN28 nucleic acid molecule is a candidate compound for treating a fungal infection, and wherein the KIN28 nucleic acid molecule is selected from the group consisting of (i) a nucleic acid molecule that encodes a polypeptide containing the amino acid sequence of SEQ ID NO: 6 or SEQ ID NO: 7; and (ii) a nucleic acid molecule that encodes a naturally occurring allelic variant of a polypeptide containing the amino acid sequence of SEQ ID NO: 6 or SEQ ID NO: 7, wherein the nucleic acid molecule hybridizes under stringent conditions to a nucleic acid molecule consisting of the nucleotide sequence of SEQ ID NO: 1, SEQ ID NO: 2, SEQ ID NO: 3, SEQ ID NO: 4, or SEQ ID NO: 5 or the complement of SEQ ID NO: 1, SEQ ID NO: 2, SEQ ID NO: 3, SEQ ID NO: 4, or SEQ ID NO: 5. The method can further include determining whether a candidate compound that binds to the KIN28 nucleic acid molecule inhibits growth of fungi, relative to growth of fungi grown in the absence of the test compound, wherein inhibition of growth indicates that the candidate compound is an antifungal agent. In one example, the Kin28 can be derived from a non-pathogenic fungus. In another example, the Kin28 is derived from a pathogenic fungus.

The test compound can be selected from the group consisting of a polypeptide, a small organic or inorganic molecule, a ribonucleic acid, a deoxyribonucleic acid, an antisense oligonucleotide, a peptomimetic, a polysaccharide, and a ribozyme. In one example, the test compound is an antisense oligonucleotide. In another example, the test compound is a ribozyme.

In another aspect, the invention features a method for identifying a candidate compound for treating a fungal infection, the method including: (a) contacting a homolog of Kin28 with a test compound; and (b) detecting binding of the test compound to the homolog of Kin28, wherein a compound that binds to the homolog of Kin28 is a candidate compound for treating a fungal infection, wherein Kin28 is selected from the group consisting of a first nucleic acid molecule which encodes either a polypeptide containing the amino acid sequence of SEQ ID NO: 6 or SEQ ID NO: 7 or a naturally occurring allelic variant of a polypeptide containing the amino acid sequence of SEQ ID NO: 6 or SEQ ID NO: 7, wherein the first nucleic acid molecule hybridizes to a second nucleic acid molecule under stringent conditions, the second nucleic acid molecule consisting of the nucleotide sequence of SEQ ID NO: 1, SEQ ID NO: 2, SEQ ID NO: 3, SEQ ID NO: 4, or SEQ ID NO: 5 or the complement of SEQ ID NO: 1, SEQ ID NO: 2, SEQ ID NO: 3, SEQ ID NO: 4, or SEQ ID NO: 5. The method can further include determining whether a candidate compound that binds to the homolog of Kin28 inhibits growth of fungi, relative to growth of fungi grown in the absence of the test compound that binds to the homolog of Kin28, wherein inhibition of growth indicates that the candidate compound is an antifungal agent. In one example, the homolog of Kin28 can be derived from a non-pathogenic fungus. In another example, the homolog of Kin28 is derived from a pathogenic fungus.

The test compound can be immobilized on a substrate, and binding of the test compound to the homolog of Kin28 can be detected as immobilization of the homolog of Kin28 on the immobilized test compound. Immobilization of the homolog of Kin28 on the test compound can be detected in an immunoassay with an antibody that specifically binds to the homolog of Kin28.

The test compound can be selected from the group consisting of a polypeptide, a small organic or inorganic molecule, a ribonucleic acid, a deoxyribonucleic acid, an antisense oligonucleotide, a peptomimetic, a polysaccharide, and a ribozyme. In one example, the test compound is an antisense oligonucleotide. In another example, the test compound is a ribozyme.

The invention also includes a method for identifying a candidate compound for the treatment of a fungal infection, the method including the steps, in sequence, of: (a) preparing a first cell and a second cell, the first and second cells being capable of expressing KIN28 ; (b) contacting the first cell with a test compound; (c) determining the level of expression of the KIN28 gene in the first and second cells; (d) comparing the level of expression of the KIN28 gene in the first cell with the level of expression of the Kin28 in the second cell; and (e) selecting the test compound as a candidate compound for treating a fungal infection, if the expression of the KIN28 gene in the first cell is less than the expression of the KIN28 gene in the second cell, wherein the KIN28 gene is a first nucleic acid molecule which encodes a polypeptide containing the amino acid sequence of SEQ ID NO: 6 or SEQ ID NO: 7, or a naturally occurring allelic variant thereof, and wherein the first nucleic acid molecule hybridizes under stringent conditions to a second nucleic acid molecule, the second nucleic acid molecule consisting of a nucleotide sequence of SEQ ID NO: 1, SEQ ID NO: 2, SEQ ID NO: 3, SEQ ID NO: 4, or SEQ ID NO: 5.

The invention also includes a method for identifying a candidate compound for the treatment of a fungal infection, the method including the steps, in sequence, of: (a) preparing a first cell and a second cell, the first and second cells being capable of expressing a homolog of KIN28; (b) contacting the first cell with a test compound; (c) determining the level of expression of the homolog of the KIN28 gene in the first and second cells; (d) comparing the level of expression of the homolog of the KIN28 gene in the first cell with the level of expression of the homolog of the KIN28 gene in the second cell; and (e) selecting the test compound as a candidate compound for treating a fungal infection, if the level of expression of the homolog of the KIN28 gene in the first cell is less than the level of expression of the homolog of the KIN28 gene in the second cell, wherein the KIN28 gene is a first nucleic acid molecule that encodes a polypeptide containing the amino acid sequence of SEQ ID NO: 6 or SEQ ID NO: 7, or a naturally occurring allelic variant thereof, and wherein the first nucleic acid molecule hybridizes under stringent conditions to a second nucleic acid molecule, the second nucleic acid molecule consisting of a nucleotide sequence of SEQ ID NO: 1, SEQ ID NO: 2, SEQ ID NO: 3, SEQ ID NO: 4, or SEQ ID NO: 5.

The invention offers several advantages. The invention provides targets, based on essential functions, for identifying potential agents for the effective treatment of opportunistic infections caused by Aspergillus and other related fungal species. Also, the methods for identifying antifungal candidates or agents can be configured for high throughput screening of numerous candidate antifungal agents.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein. All publications, patent applications, patents, and other references mentioned herein are incorporated herein by reference in their entirety. In the case of a conflict, the present specification, including definitions, will control. In addition, the materials, methods, and examples are illustrative and are not intended to limit the scope of the invention, which is defined by the claims.

Other features and advantages of the invention will be apparent from the following detailed description, and from the claims.

### BRIEF DESCRIPTION OF THE DRAWINGS

FIGS. 1A-1B are schematic representations that depict an alignment of the genomic sequence (SEQ ID NO: 1) and partial cDNA sequence (SEQ ID NO: 2) of Aspergillus nidulans KIN28. The initiation (ATG) and termination (TAG) codons are underlined.

FIGS. 2A-2B are schematic representations that depict an alignment of the genomic sequence (SEQ ID NO: 3) and cDNA sequence (SEQ ID NO: 4) of Aspergillus fumigatus KIN28. The initiation (ATG) and termination (TAA) codons are underlined.

FIG. 3 is a schematic representation that depicts an alignment of the amino acid sequences of the Kin28 protein of Aspergillus nidulans (SEQ ID NO: 6), Aspergillus fumigatus (SEQ ID NO: 7), and Saccharomyces cerevisiae (SEQ ID NO: 8).

### DETAILED DESCRIPTION OF THE INVENTION

Nucleic acids encoding Aspergillus Kin28 polypeptides have been identified and are described herein. Because KIN28 is an essential gene, the KIN28 genes and polypeptides are useful targets for identifying compounds that are, or potentially are, inhibitors of fungi, e.g., Aspergillus species such as Aspergillus nidulans and Aspergillus fumigatus, in which Kin28 polypeptides are expressed.

Nucleic Acid and Amino Acid Sequences

Nucleic acids described herein include both RNA and DNA, including genomic DNA and synthetic (e.g., chemically synthesized) DNA. Nucleic acids can be double-stranded or single-stranded. Where single-stranded, the nucleic acid can be a sense strand or an antisense strand. Nucleic acids can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases.

An isolated nucleic acid is a DNA or RNA that is not immediately contiguous with both of the coding sequences with which it is immediately contiguous (one on the 5′ end and one on the 3′ end) in the naturally occurring genome of the organism from which it is derived. Thus, in one embodiment, an isolated nucleic acid includes some or all of the 5′ non-coding (e.g., promoter) sequences that are immediately contiguous to the coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a genomic DNA fragment produced by PCR or restriction endonuclease treatment) independent of other sequences. It also includes a recombinant DNA that is part of a hybrid gene encoding an additional polypeptide sequence. The terms “isolated” and “purified” refer to a nucleic acid or polypeptide that is substantially free of cellular or viral material with which it is naturally associated, or culture medium (when produced by recombinant DNA techniques), or chemical precursors or other chemicals (when chemically synthesized). Moreover, an isolated nucleic acid fragment is a nucleic acid fragment that is not naturally occurring as a fragment and would not be found in the natural state.

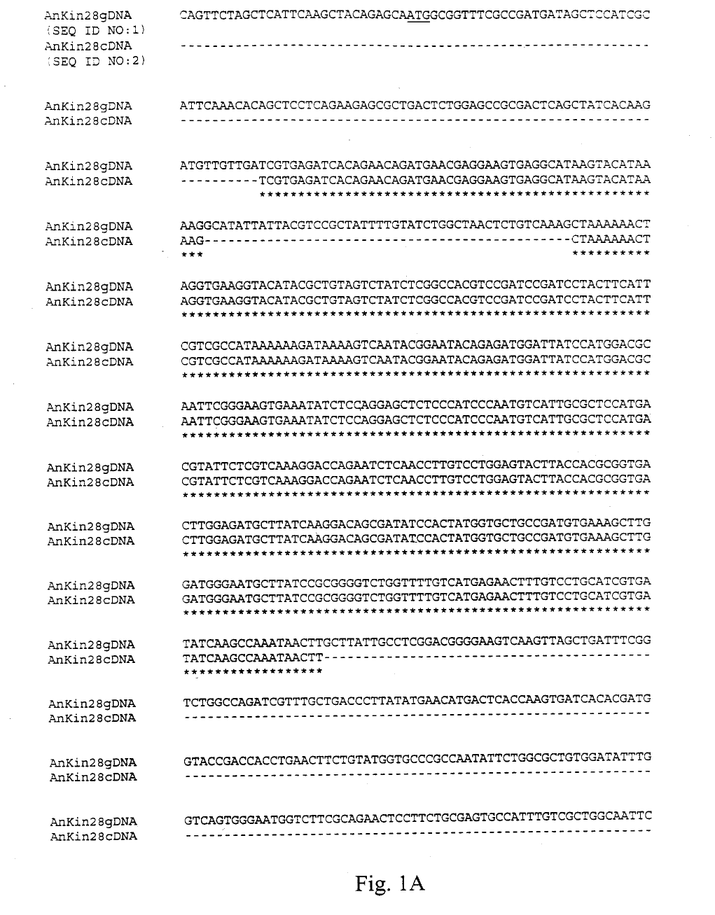

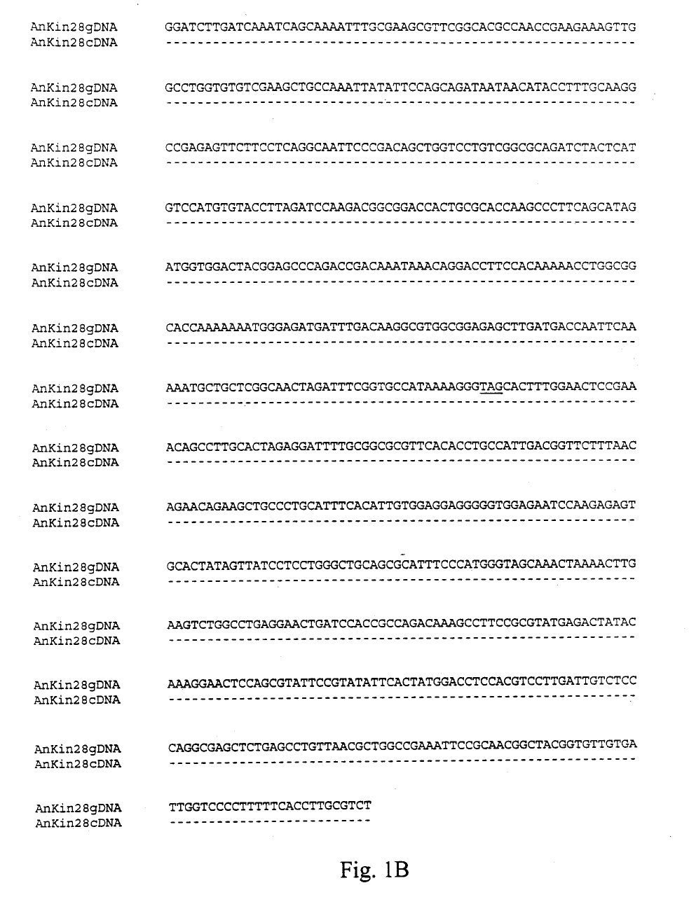

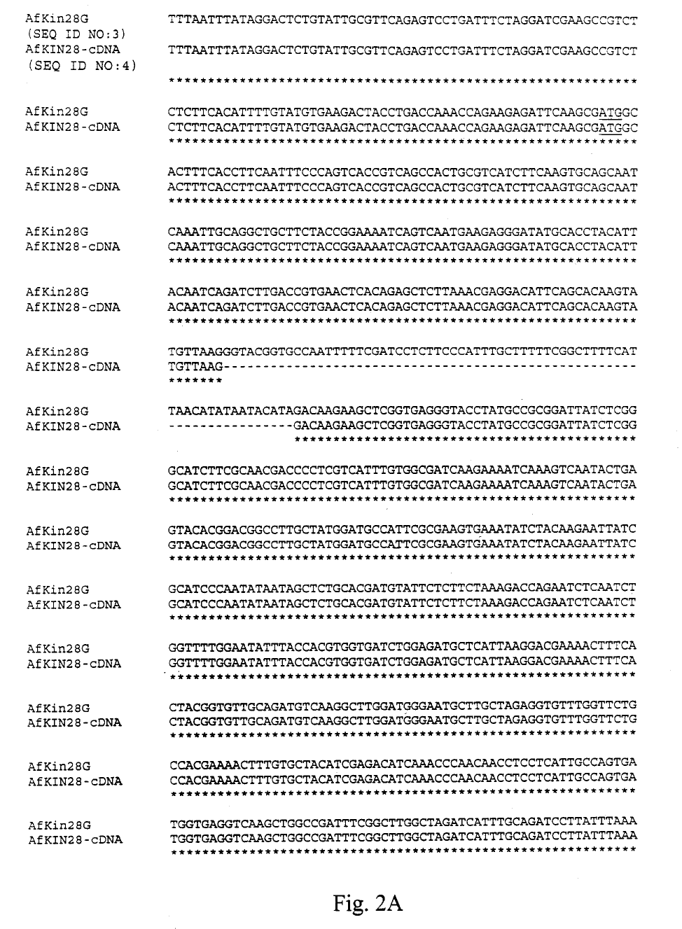

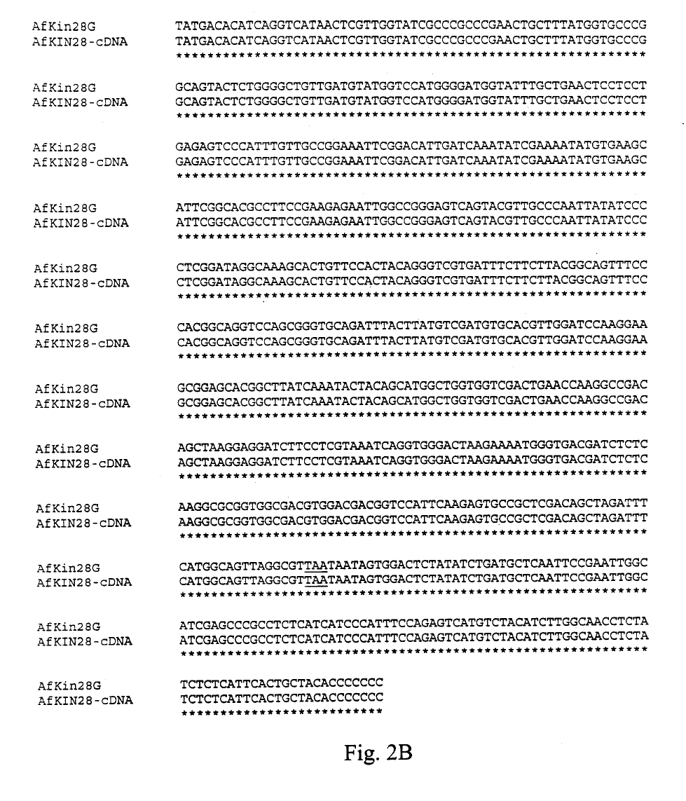

A nucleic acid sequence that is substantially identical to a KIN28 nucleotide sequence is at least 80% identical to the nucleotide sequence of KIN28 as represented by SEQ ID NO: 1, SEQ ID NO: 2, SEQ ID NO: 3, SEQ ID NO: 4, or SEQ ID NO: 5, as depicted in FIGS. 1A-1B and 2A-2B. For purposes of comparison of nucleic acids, the length of the reference nucleic acid sequence will generally be at least 40 nucleotides, e.g., at least 60 or more nucleotides.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity =# of identical positions/total # of overlapping positions x 100). Preferably, the two sequences are the same length.

The determination of percent identity or homology between two sequences can be accomplished using a mathematical algorithm. A preferred, non-limiting example of a mathematical algorithm utilized for the comparison of two sequences is the algorithm of Karlin and Altschul (1990) Proc. Nat'l Acad. Sci. USA 87:2264-2268, modified as in Karlin and Altschul (1993) Proc. Nat'l Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to KIN28 nucleic acid molecules of the invention. BLAST protein searches can be performed with the XBLAST program, score=50, wordlength=3 to obtain amino acid sequences homologous to Kin28 protein molecules of the invention. To obtain gapped alignments for comparison purposes, Gapped BLAST can be utilized as described in Altschul et al. (1997) Nucleic Acids Res. 25:3389-3402. When utilizing BLAST and Gapped BLAST programs, the default parameters of the respective programs (e.g., XBLAST and NBLAST) can be used. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, only exact matches are counted. For purposes of amino acid sequence comparison, the length of a reference Kin28 polypeptide sequence will generally be at least 16 amino acids, e.g., at least 20 or 25 amino acids.

The term “homology” as used herein can be equated with the term “identity”. Relative sequence homology (i.e., sequence identity) can be determined by commercially available computer programs that can calculate the percent homology between two or more sequences. A typical example of such a computer program is CLUSTAL.

The terms “variant,” “homolog,” or “fragment” in relation to the nucleotide sequence encoding Kin28 of the present invention include any substitution, variation, modification, replacement, deletion, or addition of one (or more) nucleotides from or to the sequence of a KIN28 gene. Typically, the resultant nucleotide sequence encodes or is capable of encoding a Kin28 polypeptide that has at least 50% of the biological activity of the referenced Kin28 polypeptide (e.g., as represented by SEQ ID NO: 6 or SEQ ID NO: 7). Kin28 biological activities are described herein and include, e.g., the ability to associate with a cyclin, e.g., Ccl1, the ability to promote RNA polymerase II-mediated transcription, and/or the ability to mediate serine/threonine kinase activity, e.g., the ability to phosphorylate the CTD of RNA polymerase II. In particular, the term “homolog” covers homology with respect to structure and/or function providing the resultant nucleotide sequence that codes for or is capable of coding for a Kin28 polypeptide that is least as biologically active as a Kin28 encoded by the sequence shown as SEQ ID NO: 1, SEQ ID NO: 2, SEQ ID NO: 3, SEQ ID NO: 4, or SEQ ID NO: 5. With respect to sequence homology, there is at least 50% (e.g., 60%, 75%, 85%, 90%, 95%, 98%, or 100%) homology to the sequence shown as SEQ ID NO: 1, SEQ ID NO: 2, SEQ ID NO: 3, SEQ ID NO: 4, or SEQ ID NO: 5.

“Substantial identity” means at least 80% sequence identity, as judged by direct sequence alignment and comparison. “Substantial identity” when assessed by the BLAST algorithm equates to sequences which match with an EXPECT value of at least about 7, e.g., at least about 9, 10, or more. The default threshold for EXPECT in BLAST searching is usually 10.

Also included within the scope of the present invention are alleles of a KIN28 gene. As used herein, an “allele” or “allelic sequence” is an alternative form of a KIN28 gene. Any given gene can have none, one, or more than one allelic form. Common mutational changes which give rise to alleles are generally ascribed to deletions, additions, or substitutions of amino acids. Each of these types of changes can occur alone, or in combination with the others, one or more times in a given sequence.

The Kin28 polypeptides of the invention include, but are not limited to, recombinant polypeptides and natural polypeptides. Also included are nucleic acid sequences that encode forms of Kin28 polypeptides in which naturally occurring amino acid sequences are altered or deleted. Preferred nucleic acids encode polypeptides that are soluble under normal physiological conditions. Also within the invention are nucleic acids encoding fusion proteins in which a portion of the Kin28 polypeptide is fused to an unrelated polypeptide (e.g., a marker polypeptide or a fusion partner) to create a fusion protein. For example, the polypeptide can be fused to a hexa-histidine tag to facilitate purification of bacterially expressed polypeptides, or to a hemagglutinin tag to facilitate purification of polypeptides expressed in eukaryotic cells. The invention also includes, for example, isolated polypeptides (and the nucleic acids that encode these polypeptides) that include a first portion and a second portion; the first portion includes, e.g., a Kin28 polypeptide, and the second portion includes an immunoglobulin constant (Fc) region or a detectable marker.

The fusion partner can be, for example, a polypeptide that facilitates secretion, e.g., a secretory sequence. Such a fused polypeptide is typically referred to as a preprotein. The secretory sequence can be cleaved by the host cell to form the mature protein. Also within the invention are nucleic acids that encode a Kin28 polypeptide fused to a polypeptide sequence to produce an inactive preprotein. Preproteins can be converted into the active form of the protein by removal of the inactivating sequence.

The invention also includes nucleic acids that hybridize, e.g., under stringent hybridization conditions (as defined herein) to all or a portion of the nucleotide sequences represented by SEQ ID NO: 1, SEQ ID NO: 2, SEQ ID NO: 3, SEQ ID NO: 4, or SEQ ID NO: 5, or its complement. The hybridizing portion of the hybridizing nucleic acids is typically at least 16 (e.g., 20, 30, or 50) nucleotides in length. The hybridizing portion of the hybridizing nucleic acid is at least 50%, e.g., at least 60%, 70%, 80%, 95%, or at least 98% or 100%, identical to the sequence of a portion or all of a nucleic acid encoding a Kin28 polypeptide or its complement. Hybridizing nucleic acids of the type described herein can be used as a cloning probe, a primer (e.g., a PCR primer), or a diagnostic probe. Nucleic acids that hybridize to the nucleotide sequence represented by SEQ ID NO: 1, SEQ ID NO: 2, SEQ ID NO: 3, SEQ ID NO: 4, or SEQ ID NO: 5 are considered “antisense oligonucleotides.”

In one aspect, this invention provides isolated nucleic acid molecules encoding Kin28 polypeptides or biologically active portions thereof, as well as nucleic acid fragments suitable as primers or hybridization probes for the detection of Kin28-encoding nucleic acids (e.g., fragments of at least 15 nucleotides (e.g., at least 18, 20, 25, 30, 35, 45, 60, 80, or 100 nucleotides)).

The invention features a nucleic acid molecule that is at least 50% (or 65%, 75%, 85%, 95%, 98%, or 100%) identical to the nucleotide sequence shown in SEQ ID NO: 1, SEQ ID NO: 2, SEQ ID NO: 3, SEQ ID NO: 4, SEQ ID NO: 5, the nucleotide sequence of the cDNA insert of the plasmid deposited with ATCC as Accession Number______ (the “cDNA of ATCC______”), the nucleotide sequence of the cDNA inse plasmid deposited with ATCC as Accession Number______(the “cDNA of ATCC ______”), or a complement thereof. The invention features a nucleic acid molecule that includes a fragment of at least 50 (e.g., 100, 150, 200, 300, 325, 350, 375, 400, 425, 450, 500, 550, 600, 650, 700, 800, 900, 1000, 1200, 1400, 1600, or 1640) nucleotides of the nucleotide sequence shown in SEQ ID NO: 1, SEQ ID NO: 2, SEQ ID NO: 3, SEQ ID NO: 4, SEQ ID NO: 5, the nucleotide sequence of the cDNA ATCC______, the nucleotide sequence of the cDNA ATCC______, or a complement thereof.

The invention also features a nucleic acid molecule that includes a nucleotide sequence encoding a polypeptide having an amino acid sequence that is at least 45% (or 55%, 65%, 75%, 85%, 95%, 98%, or 100%) identical to the amino acid sequence of SEQ ID NO: 6, SEQ ID NO: 7, the amino acid sequence encoded by the cDNA of ATCC______, or the amino acid sequence encoded by the cDNA of ATCC______.

Also within the invention is a nucleic acid molecule that encodes a fragment of a polypeptide having the amino acid sequence of SEQ ID NO: 6 or SEQ ID NO: 7, the fragment including at least 17 (e.g., 25, 30, 50, 100, 150, 300, or 400) contiguous amino acids of SEQ ID NO: 6, SEQ ID NO: 7, the amino acid sequence encoded by the cDNA of ATCC______, or the amino acid sequence encoded by the cDNA of ATCC______.

Another embodiment of the invention features KIN28 nucleic acid molecules that specifically detect Aspergillus nucleic acid molecules, e.g., Aspergillus nidulans and/or Aspergillus fumigatus nucleic acid molecules, in a sample containing nucleic acid molecules encoding other proteins. For example, in one embodiment, a KIN28 nucleic acid molecule hybridizes under stringent conditions to a nucleic acid molecule that includes the nucleotide sequence of SEQ ID NO: 1, SEQ ID NO: 2, SEQ ID NO: 3, SEQ ID NO: 4, SEQ ID NO: 5, the nucleotide sequence of the cDNA ATCC, the nucleotide sequence of the cDNA ATCC______, or a complement thereof. In another embodiment, the KIN28 nucleic acid molecule is at least 50 (e.g., 100, 200, 300, 400, 500, 700, 900, 1100, 1300, or 1600) nucleotides in length and hybridizes under stringent conditions to a nucleic acid molecule that includes the nucleotide sequence shown in SEQ ID NO: 1, SEQ ID NO: 2, SEQ ID NO: 3, SEQ ID NO: 4, SEQ ID NO: 5, the nucleotide sequence of the cDNA ATCC______, the nucleotide sequence of the cDNA ATCC______, or a complement thereof. In another embodiment, the invention provides an isolated nucleic acid molecule which is antisense to the coding strand of a KIN28 nucleic acid.

Also useful in the invention are various engineered cells, e.g., transformed host cells, that contain a KIN28 nucleic acid described herein. A transformed cell is a cell into which (or into an ancestor of which) has been introduced, by means of recombinant DNA techniques, a nucleic acid encoding a Kin28 polypeptide. Both prokaryotic and eukaryotic cells are included, e.g., fungi and bacteria, such as E. coli, and the like.

Also useful in the invention are genetic constructs (e.g., vectors and plasmids) that include a nucleic acid of the invention operably linked to a transcription and/or translation sequence to enable expression, e.g., expression vectors. A selected nucleic acid, e.g., a DNA molecule encoding a Kin28 polypeptide, is “operably linked” when it is positioned with respect to one or more controlling sequence elements, e.g., a promoter, so that the controlling sequence elements can direct transcription and/or translation of the selected nucleic acid. For example, the selected nucleic acid can be positioned adjacent to the controlling sequence elements.

In another aspect, the invention provides a vector, e.g., a recombinant expression vector, that includes a KIN28 nucleic acid molecule of the invention. In another embodiment the invention provides a host cell containing such a vector. The invention also provides a method for producing a Kin28 polypeptide by culturing, in a suitable medium, a host cell of the invention containing a recombinant expression vector such that a Kin28 polypeptide is produced.

The invention also features purified or isolated Kin28 polypeptides. The terms “protein” and “polypeptide” both refer to any chain of amino acids, regardless of length or post-translational modification (e.g., glycosylation or phosphorylation). Thus, the term Kin28 polypeptide includes full-length, naturally occurring, isolated Kin28 proteins, as well as recombinantly or synthetically produced polypeptides that correspond to the full-length, naturally occurring proteins, or to a portion of the naturally occurring or synthetic polypeptide.

Kin28 polypeptides possess at least one biological activity possessed by a naturally occurring Kin28 protein, e.g., the ability to associate with a cyclin, e.g., Ccl1, the ability to promote RNA polymerase II-mediated transcription, and/or the ability to mediate serine/threonine kinase activity, e.g., the ability to phosphorylate the C-terminal repeat domain (CTD) of RNA polymerase II. It is not necessary that the Kin28 polypeptide have an activity that is equivalent to that of a naturally occurring Kin28. For example, the Kin28 polypeptide can have 20, 50, 75, 90, 100, or an even higher percent of the wild-type activity, e.g., a kinase activity.

A purified or isolated compound is a composition that is at least 60% by weight the compound of interest, e.g., a Kin28 polypeptide or antibody. The composition can be of higher purity, e.g., at least 75% (e.g., at least 90%, 95%, or even 99%) by weight the compound of interest. Purity can be measured by any appropriate standard method, e.g., column chromatography, polyacrylamide gel electrophoresis, or HPLC analysis.

In the case of polypeptide sequences that are less than 100% identical to a reference sequence, the non-identical positions are preferably, but not necessarily, conservative substitutions for the reference sequence. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a Kin28 polypeptide is preferably replaced with another amino acid residue from the same side chain family. Alternatively, in another embodiment, mutations can be introduced randomly along all or part of a Kin28 coding sequence, such as by saturation mutagenesis, and the resultant mutants can be screened for Kin28 biological activity to identify mutants that retain activity. Following mutagenesis, the encoded protein can be expressed recombinantly and the activity of the protein can be determined.

Where a particular polypeptide is said to have a specific percent identity to a reference polypeptide of a defined length, the percent identity is relative to the reference polypeptide. Thus, a polypeptide that is 50% identical to a reference polypeptide that is 100 amino acids long can be a 50 amino acid polypeptide that is completely identical to a 50 amino acid long portion of the reference polypeptide. It also might be a 100 amino acid long polypeptide which is 50% identical to the reference polypeptide over its entire length. Of course, other polypeptides also will meet the same criteria.

In other embodiments, the invention features: an isolated Kin28 protein having an amino acid sequence that is at least about 45% (e.g., 55%, 65%, 75%, 85%, 95%, 98%, or 100%) identical to the amino acid sequence of SEQ ID NO: 6 or SEQ ID NO: 7; an isolated Kin28 protein which is encoded by a nucleic acid molecule having a nucleotide sequence that is at least about 50% (e.g., 60%, 75%, 85%, 95%, or 100%) identical to SEQ ID NO: 1, SEQ ID NO: 2, SEQ ID NO: 3, SEQ ID NO: 4, SEQ ID NO: 5, the nucleotide sequence of the cDNA ATCC_____, the nucleotide sequence of the cDNA ATCC_____; and an isolated Kin28 protein which is encoded by a nucleic acid molecule having a nucleotide sequence which hybridizes under stringent hybridization conditions (as defined herein) to a nucleic acid molecule having the nucleotide sequence of SEQ ID NO: 1, SEQ ID NO: 2, SEQ ID NO: 3, SEQ ID NO: 4, or SEQ ID NO: 5, or the non-coding strand of the cDNA of ATCC_____ or of the cDNA of ATCC_____.

The invention also features purified or isolated antibodies that specifically bind to a Kin28 polypeptide, e.g., an Aspergillus Kin28 polypeptide. An antibody “specifically binds” to a particular antigen, e.g., a Kin28 polypeptide, when it binds to that antigen, but does not recognize and bind to other molecules in a sample, e.g., a biological sample, that naturally includes a Kin28 polypeptide. In addition, an antibody specifically binds to a Aspergillus Kin28 polypeptide when it does not substantially bind to Kin28 polypeptides from other genera (e.g., Saccharomyces), particularly Kin28 polypeptides of an organism to be treated by the methods of the invention (e.g., humans or domesticated animals).

Aspergillus nidulans and Asperzillus fumigatus KIN28 Genes and cDNAs

FIGS. 1A-1B depict an alignment of the genomic sequence (SEQ ID NO: 1) and partial cDNA sequence (SEQ ID NO: 2) of Aspergillus nidulans KIN28. The portion of the genomic sequence corresponding to an intron is absent in the cDNA sequence and is represented by dashes in the alignment. The initiation (ATG) and termination (TAG) codons are underlined.

FIGS. 2A-2B depict an alignment of the genomic sequence (SEQ ID NO: 3) and cDNA sequence (SEQ ID NO: 4) of Aspergillus fumigatus KIN28. The portion of the genomic sequence corresponding to an intron is absent in the cDNA sequence and is represented by dashes in the alignment. The initiation (ATG) and termination (TAA) codons are underlined. The open reading frame of Aspergillus fumigatus KIN28 extends from the initiation codon to the nucleotide immediately before the termination codon of SEQ ID NO: 4 (SEQ ID NO: 5).

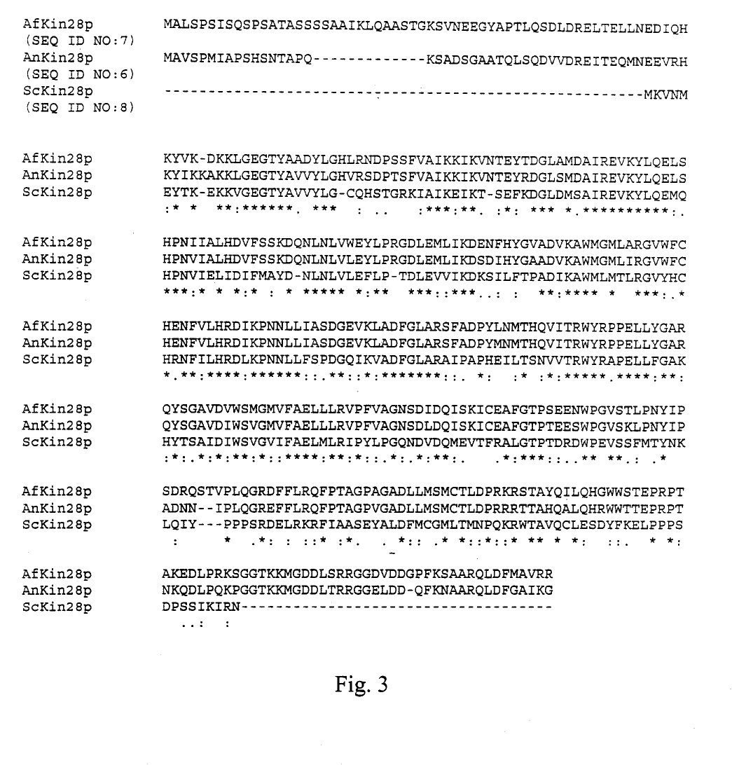

FIG. 3 depicts an alignment of the amino acid sequences of the Kin28 protein of Aspergillus nidulans (SEQ ID NO: 6), Aspergillus fumigatus (SEQ ID NO: 7), and Saccharomyces cerevisiae (SEQ ID NO: 8). A star at a position below the alignment indicates that the amino acid residue is conserved among the Kin28 proteins of the three species.

Identification of KIN28 Genes in Additional Fungal Strains

Since specific Aspergillus KIN28 genes have been identified, these genes, or fragments thereof, can be used to detect homologous genes in other organisms, including other species of Aspergillus. Fragments of a nucleic acid (DNA or RNA) encoding a Kin28 polypeptide (or sequences complementary thereto) can be used as probes in conventional nucleic acid hybridization assays of various organisms. For example, nucleic acid probes (which typically are 8-30, or usually 15-20, nucleotides in length) can be used to detect KIN28 genes using standard molecular biology methods, such as Southern blotting, Northern blotting, dot or slot blotting, PCR amplification methods, colony hybridization methods, and the like. Typically, an oligonucleotide probe based on the nucleic acid sequences described herein, or fragment thereof, is labeled and used to screen a genomic library constructed from mRNA obtained from a fungal strain of interest. A suitable method of labeling involves using polynucleotide KIN28 to add ³²P-labeled ATP to the oligonucleotide used as the probe. This method is well known in the art, as are several other suitable methods (e.g., biotinylation and enzyme labeling).

Hybridization of the oligonucleotide probe to the library, or other nucleic acid sample, typically is performed under moderate to high stringency conditions. Nucleic acid duplex or hybrid stability is expressed as the melting temperature or Tm, which is the temperature at which a probe dissociates from a target DNA. This melting temperature is used to define the required stringency conditions. If sequences are to be identified that are related and substantially identical to the probe, rather than identical, then it is useful to first establish the lowest temperature at which only homologous hybridization occurs with a particular concentration of salt (e.g., SSC or SSPE). Then, assuming that 1% mismatching results in a 1° C. decrease in the Tm, the temperature of the final wash in the hybridization reaction is reduced accordingly (for example, if sequences having >95% identity with the probe are sought, the final wash temperature is decreased by 5° C.). In practice, the change in Tm can be between 0.5° C. and 1.5° C. per 1% mismatch.

High stringency conditions include hybridizing at 68° C. in 5×SSC/5×Denhardt's solution/1.0% SDS, or in 0.5 M NaHPO₄ (pH 7.2)/1 mM EDTA/7% SDS, or in 50% formamide/0.25 M NaHPO₄ (pH 7.2)/0.25 M NaCl/1 mM EDTA/7% SDS; and washing in 0.2×SSC/0.1% SDS at room temperature or at 42° C., or in 0.1×SSC/0.1% SDS at 68° C., or in 40 mM NaHPO₄ (pH 7.2)/1 mM EDTA/5% SDS at 50° C., or in 40 mM NaHPO₄ (pH 7.2) 1 mM EDTA/1% SDS at 50° C. Stringent conditions include washing in 3×SSC at 42° C. The parameters of salt concentration and temperature can be varied to achieve the optimal level of identity between the probe and the target nucleic acid. Additional guidance regarding such conditions is available in the art, for example, in Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

In one approach, libraries constructed from pathogenic or non-pathogenic fungal strains are screened. For example, such strains can be screened for expression of a KIN28 gene of the invention by Northern blot analysis. Upon detection of transcripts of a KIN28 gene, libraries can be constructed from RNA isolated from the appropriate strain, utilizing standard techniques well known to those of skill in the art. Alternatively, a total genomic DNA library can be screened using a KIN28 gene probe.

New gene sequences can be isolated, for example, by performing PCR using two degenerate oligonucleotide primer pools designed on the basis of nucleotide sequences within a KIN28 gene as depicted herein. The template for the reaction can be DNA obtained from strains known or suspected to express the KIN28 gene of the invention. The PCR product can be subcloned and sequenced.

Synthesis of the various Kin28 polypeptides (or an antigenic fragment thereof) for use as antigens, or for other purposes, can be accomplished using any of the various art-known techniques. For example, a Kin28 polypeptide, or an antigenic fragment(s), can be synthesized chemically in vitro, or enzymatically (e.g., by in vitro transcription and translation). Alternatively, the gene can be expressed in, and the polypeptide purified from, a cell (e.g., a cultured cell) by using any of the numerous, available gene expression systems. For example, the polypeptide antigen can be produced in a prokaryotic host (e.g., E. coli) or in eukaryotic cells, such as yeast cells.

Proteins and polypeptides can also be produced in plant cells, if desired. For plant cells, viral expression vectors (e.g., cauliflower mosaic virus and tobacco mosaic virus) and plasmid expression vectors (e.g., Ti plasmid) are suitable. Such cells are available from a wide range of sources (e.g., the American Type Culture Collection, Manassas, Va.; also, see, e.g., Ausubel et al., Current Protocols in Molecular Biology, John Wiley & Sons, New York, 1994). The optimal methods of transformation or transfection and the choice of expression vehicle will depend on the host system selected. Transformation and transfection methods are described, e.g., in Ausubel et al., supra; expression vehicles can be chosen from those provided, e.g., in Cloning Vectors: A Laboratory Manual (P.H. Pouwels et al., 1985, Supp. 1987). The host cells harboring the expression vehicle can be cultured in conventional nutrient media, adapted as needed for activation of a chosen gene, repression of a chosen gene, selection of transformants, or amplification of a chosen gene.

If desired, the Kin28 polypeptide can be produced as a fusion protein. For example, the expression vector pUR278 (Ruther et al., EMBO J., 2:1791, 1983) can be used to create lacZ fusion proteins. Known pGEX vectors can be used to express foreign polypeptides as fusion proteins with glutathione S-transferase (GST). In general, such fusion proteins are soluble and can be easily purified from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

In an exemplary expression system, a baculovirus such as Autographa californica nuclear polyhedrosis virus (AcNPV), which grows in Spodoptera frugiperda cells, can be used as a vector to express foreign genes. A coding sequence encoding a Kin28 polypeptide can be cloned into a non-essential region (for example the polyhedrin gene) of the viral genome and placed under control of a promoter, e.g., the polyhedrin promoter or an exogenous promoter. Successful insertion of a gene encoding a Kin28 polypeptide can result in inactivation of the polyhedrin gene and production of non-occluded recombinant virus (i.e., virus lacking the proteinaceous coat encoded by the polyhedrin gene). These recombinant viruses are then typically used to infect insect cells (e.g., Spodoptera frugiperda cells) in which the inserted gene is expressed (see, e.g., Smith et al., J. Virol., 46:584, 19183; Smith, U.S. Pat. No. 4,215,051). If desired, mammalian cells can be used in lieu of insect cells, provided that the virus is engineered such that the gene encoding the Kin28 polypeptide is placed under the control of a promoter that is active in mammalian cells.

In mammalian host cells, a number of viral-based expression systems can be utilized. When an adenovirus is used as an expression vector, the nucleic acid sequence encoding a Kin28 polypeptide can be ligated to an adenovirus transcription/translation control complex, e.g., the late promoter and tripartite leader sequence. This chimeric gene can then be inserted into the adenovirus genome by in vitro or in vivo recombination. Insertion into a non-essential region of the viral genome (e.g., region E1 or E3) will result in a recombinant virus that is viable and capable of expressing a Kin28 gene product in infected hosts (see, e.g., Logan, Proc. Natl. Acad. Sci. USA, 81:3655, 1984).

Specific initiation signals can be included for efficient translation of inserted nucleic acid sequences. These signals include the ATG initiation codon and adjacent sequences. In general, exogenous translational control signals, including, perhaps, the ATG initiation codon, should be provided. Furthermore, the initiation codon must be in phase with the reading frame of the desired coding sequence to ensure translation of the entire sequence. These exogenous translational control signals and initiation codons can be of a variety of origins, both natural and synthetic. The efficiency of expression can be enhanced by the inclusion of appropriate transcription enhancer elements, or transcription terminators (Bittner et al., Methods in Enzymol., 153:516, 1987).

A Kin28 polypeptide can be expressed individually or as a fusion with a heterologous polypeptide, such as a signal sequence or other polypeptide having a specific cleavage site at the N-and/or C-terminus of the protein or polypeptide. The heterologous signal sequence selected should be one that is recognized and processed, i.e., cleaved by a signal peptidase, by the host cell in which the fusion protein is expressed.

A host cell can be chosen that modulates the expression of the inserted sequences, or modifies and processes the gene product in a specific, desired fashion. Such modifications and processing (e.g., cleavage) of protein products can facilitate optimal functioning of the protein. Various host cells have characteristic and specific mechanisms for post-translational processing and modification of proteins and gene products. Appropriate cell lines or host systems familiar to those of skill in the art of molecular biology can be chosen to ensure the correct modification and processing of the foreign protein expressed. To this end, eukaryotic host cells that possess the cellular machinery for proper processing of the primary transcript, and phosphorylation of the gene product can be used. Such mammalian host cells include, but are not limited to, CHO, VERO, BHK, HeLa, COS, MDCK, 293, 3T3, WI38, and choroid plexus cell lines.

If desired, the Kin28 polypeptide can be produced by a stably-transfected mammalian cell line. A number of vectors suitable for stable transfection of mammalian cells are available to the public, see, e.g., Pouwels et al. (supra); methods for constructing such cell lines are also publicly known, e.g., in Ausubel et al. (supra). In one example, DNA encoding the protein is cloned into an expression vector that includes the dihydrofolate reductase (DHFR) gene. Integration of the plasmid and, therefore, the gene encoding the Kin28 polypeptide into the host cell chromosome is selected for by including 0.01-300 μM methotrexate in the cell culture medium (as described in Ausubel et al., supra). This dominant selection can be accomplished in most cell types.

Recombinant protein expression can be increased by DHFR-mediated amplification of the transfected gene. Methods for selecting cell lines bearing gene amplifications are described in Ausubel et al. (supra); such methods generally involve extended culture in medium containing gradually increasing levels of methotrexate. DHFR-containing expression vectors commonly used for this purpose include pCVSEII-DHFR and pAdD26SV(A) (described in Ausubel et al., supra).

A number of other selection systems can be used, including but not limited to, herpes simplex virus thymidine kinase genes, hypoxanthine-guanine phosphoribosyltransferase genes, and adenine phosphoribosyltransferase genes, which can be employed in tk, hgprt, or aprt cells, respectively. In addition, gpt, which confers resistance to mycophenolic acid (Mulligan et al., Proc. Natl. Acad. Sci. USA, 78:2072, 1981); neo, which confers resistance to the aminoglycoside G-418 (Colberre-Garapin et al., J. Mol. Biol., 150:1, 1981); and hygro, which confers resistance to hygromycin (Santerre et al., Gene, 30:147, 1981), can be used.

Alternatively, any fusion protein can be purified by utilizing an antibody or other molecule that specifically bind to the fusion protein being expressed. For example, a system described in Janknecht et al., Proc. Natl. Acad. Sci. USA, 88:8972 (1981), allows for the ready purification of non-denatured fusion proteins expressed in human cell lines. In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺ nitriloacetic acid-agarose columns, and histidine-tagged proteins are selectively eluted with imidazole-containing buffers.

Alternatively, a Kin28 polypeptide, or a portion thereof, can be fused to an immunoglobulin Fc domain. Such a fusion protein can be purified using a protein A column, for example. Moreover, such fusion proteins permit the production of a chimeric form of a Kin28 polypeptide having increased stability in vivo.

Once the recombinant Kin28 polypeptide is expressed, it can be isolated (i.e., purified). Secreted forms of the polypeptides can be isolated from cell culture media, while non-secreted forms must be isolated from the host cells. Polypeptides can be isolated by affinity chromatography. For example, an anti-Kin28 antibody (e.g., produced as described herein) can be attached to a column and used to isolate the protein. Lysis and fractionation of cells harboring the protein prior to affinity chromatography can be performed by standard methods (see, e.g., Ausubel et al., supra). Alternatively, a fusion protein can be constructed and used to isolate a Kin28 polypeptide (e.g., a Kin28-maltose binding fusion protein, a Kin28-é-galactosidase fusion protein, or a Kin28-trpE fusion protein; see, e.g., Ausubel et al., supra; New England Biolabs Catalog, Beverly, Mass.). The recombinant protein can, if desired, be further purified, e.g., by high performance liquid chromatography using standard techniques (see, e.g., Fisher, Laboratory Techniques In Biochemistry And Molecular Biology, eds., Work and Burdon, Elsevier, 1980).

Given the amino acid sequences described herein, polypeptides useful in practicing the invention, particularly fragments of Kin28, can be produced by standard chemical synthesis (e.g., by the methods described in Solid Phase Peptide Synthesis, 2nd ed., The Pierce Chemical Co., Rockford, Ill., 1984) and used as antigens, for example.

Antibodies

The Kin28 polypeptides (or antigenic fragments or analogs of such polypeptides) can be used to raise antibodies useful in the invention, and such polypeptides can be produced by recombinant or peptide synthetic techniques (see, e.g., Solid Phase Peptide Synthesis, supra; Ausubel et al., supra). In general, the polypeptides can be coupled to a carrier protein, such as KLH, as described in Ausubel et al., supra, mixed with an adjuvant, and injected into a host mammal. A “carrier” is a substance that confers stability on, and/or aids or enhances the transport or immunogenicity of, an associated molecule. Antibodies can be purified, for example, by affinity chromatography methods in which the polypeptide antigen is immobilized on a resin.

In particular, various host animals can be immunized by injection of a polypeptide of interest. Examples of suitable host animals include rabbits, mice, guinea pigs, and rats. Various adjuvants can be used to increase the immunological response, depending on the host species, including but not limited to Freund's (complete and incomplete adjuvant), adjuvant mineral gels such as aluminum hydroxide, surface active substances such as lysolecithin, pluronic polyols, polyanions, peptides, oil emulsions, keyhole limpet hemocyanin, dinitrophenol, BCG (bacille Calmette-Guerin), and Corynebacterium parvum. Polyclonal antibodies are heterogeneous populations of antibody molecules derived from the sera of the immunized animals.

Antibodies useful in the invention include monoclonal antibodies, polyclonal antibodies, humanized or chimeric antibodies, single chain antibodies, Fab fragments, F(ab′)₂ fragments, and molecules produced using a Fab expression library.

Monoclonal antibodies (mAbs), which are homogeneous populations of antibodies to a particular antigen, e.g., a Kin28 polypeptide, can be prepared using standard hybridoma technology (see, e.g., Kohler et al., Nature, 256:495, 1975; Kohler et al., Eur. J. Immunol., 6:511, 1976; Kohler et al., Eur. J. Immunol., 6:292, 1976; Hammerling et al., In: Monoclonal Antibodies and T Cell Hybridomas, Elsevier, N.Y., 1981; Ausubel et al., supra).

In particular, monoclonal antibodies can be obtained by any technique that provides for the production of antibody molecules by continuous cell lines in culture, such as those described in Kohler et al., Nature, 256:495, 1975; U.S. Pat. No. 4,376,110; the human B-cell hybridoma technique (Kosbor et al., Immunology Today, 4:72, 1983; Cole et al., Proc. Natl. Acad. Sci. USA, 80:2026, 1983); and the EBV-hybridoma technique (Cole et al., Monoclonal Antibodies and Cancer Therapy, Alan R. Liss, Inc., pp. 77-96, 1983). Such antibodies can be of any immunoglobulin class including IgG, IgM, IgE, IgA, IgD, and any subclass thereof. The hybridomas producing the mAbs of this invention can be cultivated in vitro or in vivo.

Once produced, polyclonal or monoclonal antibodies are tested for specific recognition of a Kin28 polypeptide in an immunoassay, such as a Western blot or immunoprecipitation analysis using standard techniques, e.g., as described in Ausubel et al., supra. Antibodies that specifically bind to a Kin28 polypeptide, or conservative variants are useful in the invention. For example, such antibodies can be used in an immunoassay to detect a Kin28 polypeptide in pathogenic or non-pathogenic strains of fungi.

Preferably, antibodies of the invention are produced using fragments of Kin28 that appear likely to be antigenic, by criteria such as high frequency of charged residues. In one specific example, such fragments are generated by standard techniques of PCR, and are then cloned into the pGEX expression vector (Ausubel et al., supra). Fusion proteins are expressed in E. coli and purified using a glutathione agarose affinity matrix as described in Ausubel, et al., supra.

If desired, several (e.g., two or three) fusions can be generated for each protein, and each fusion can be injected into at least two rabbits. Antisera can be raised by injections in a series, typically including at least three booster injections. Typically, the antisera is checked for its ability to immunoprecipitate a recombinant Kin28 polypeptide, or unrelated control proteins, such as glucocorticoid receptor, chloramphenicol acetyltransferase, or luciferase.

Techniques developed for the production of “chimeric antibodies” (Morrison et al., Proc. Natl. Acad. Sci., 81:6851, 1984; Neuberger et al., Nature, 312:604, 1984; Takeda et al., Nature, 314:452, 1984) can be used to splice the genes from a mouse antibody molecule of appropriate antigen specificity together with genes from a human antibody molecule of appropriate biological activity. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. Nos. 4,946,778 and 4,704,692) can be adapted to produce single chain antibodies against a Kin28 polypeptide. Single chain antibodies are formed by linking the heavy and light chain fragments of the Fv region via an amino acid bridge, resulting in a single chain polypeptide.

Antibody fragments that recognize and bind to specific epitopes can be generated by known techniques. For example, such fragments can include but are not limited to F(ab′)₂ fragments, which can be produced by pepsin digestion of the antibody molecule, and Fab fragments, which can be generated by reducing the disulfide bridges of F(ab′)₂ fragments. Alternatively, Fab expression libraries can be constructed (Huse et al., Science, 246:1275, 1989) to allow rapid and easy identification of monoclonal Fab fragments with the desired specificity.

Polyclonal and monoclonal antibodies that specifically bind to a Kin28 polypeptide can be used, for example, to detect expression of Kin28 in another strain of fungi. For example, a Kin28 polypeptide can be detected in conventional immunoassays of fungal cells or extracts. Examples of suitable assays include, without limitation, Western blotting, ELISAs, radioimmune assays, and the like.

Assays for Antifungal Agents

The invention provides methods for identifying antifungal agents. In preferred methods, screening for potential or candidate antifungal agents is accomplished by identifying those compounds (e.g., small organic molecules) that bind to Kin28 and/or inhibit the activity of a Kin28 polypeptide or the expression of a KIN28 gene. For example, screens can be performed that identify those compounds that inhibit a Kin28 activity described herein, e.g., the ability to associate with a cyclin, e.g., Ccl1, the ability to promote RNA polymerase II-mediated transcription, and/or the ability to mediate serine/threonine kinase activity, e.g., the ability to phosphorylate the CTD of RNA polymerase II. Because the Saccharomyces cerevisiae KIN28 gene is an essential gene, compounds that inhibit Aspergillus Kin28 activity in such assays are expected to be antifungal agents and can be further tested, if desired, in conventional susceptibility assays.

In various suitable methods, screening for antifungal agents can be accomplished by (i) identifying those compounds that bind to Kin28 (and are thus candidate antifungal compounds) and (ii) further testing such candidate compounds for their ability to inhibit fungal growth in vitro or in vivo, in which case they are antifungal agents.

Specific binding of a test compound to a polypeptide can be detected, for example, in vitro by reversibly or irreversibly immobilizing the test compound(s) on a substrate, e.g., the surface of a well of a 96-well polystyrene microtitre plate. Methods for immobilizing polypeptides and other small molecules are well known in the art. For example, the microtitre plates can be coated with a Kin28 polypeptide by adding the polypeptide in a solution (typically, at a concentration of 0.05 to 1 mg/ml in a volume of 1-100 μl) to each well, and incubating the plates at room temperature to 37° C. for 0.1 to 36 hours. Polypeptides that are not bound to the plate can be removed by shaking the excess solution from the plate, and then washing the plate (once or repeatedly) with water or a buffer. Typically, the polypeptide is in water or a buffer. The plate is then washed with a buffer that lacks the bound polypeptide. To block the free protein-binding sites on the plates, the plates are blocked with a protein that is unrelated to the bound polypeptide. For example, 300 μl of bovine serum albumin (BSA) at a concentration of 2 mg/ml in Tris-HCl is suitable. Suitable substrates include those substrates that contain a defined cross-linking chemistry (e.g., plastic substrates, such as polystyrene, styrene, or polypropylene substrates from Corning Costar Corp., Cambridge, Mass., for example). If desired, a beaded particle, e.g., beaded agarose or beaded Sepharose, can be used as the substrate. The Kin28 is then added to the coated plate and allowed to bind to the test compound (e.g., at 37° C. for 0.5-12 hours). The plate then is rinsed as described above.

Binding of the test compound to the Kin28 can be detected by any of a variety of known methods. For example, an antibody that specifically binds to a Kin28 polypeptide can be used in an immunoassay. If desired, the antibody can be labeled (e.g., fluorescently or with a radioisotope) and detected directly (see, e.g., West and McMahon, J. Cell Biol. 74:264, 1977). Alternatively, a second antibody can be used for detection (e.g., a labeled antibody that binds to the Fc portion of an anti-YphC antibody). In an alternative detection method, the Kin28 polypeptide is labeled, and the label is detected (e.g., by labeling a Kin28 polypeptide with a radioisotope, fluorophore, chromophore, or the like). In still another method, the Kin28 polypeptide is produced as a fusion protein with a protein that can be detected optically, e.g., using green fluorescent protein (which can be detected under UV light). In an alternative method, the polypeptide can be produced as a fusion protein with an enzyme having a detectable enzymatic activity, such as horseradish peroxidase, alkaline phosphatase, é-galactosidase, or glucose oxidase. Genes encoding all of these enzymes have been cloned and are available for use by those of skill in the art. If desired, the fusion protein can include an antigen, and such an antigen can be detected and measured with a polyclonal or monoclonal antibody using conventional methods. Suitable antigens include enzymes (e.g., horseradish peroxidase, alkaline phosphatase, and é-galactosidase) and non-enzymatic polypeptides (e.g., serum proteins, such as BSA and globulins, and milk proteins, such as caseins).

In various in vivo methods for identifying polypeptides that bind to Kin28, the conventional two-hybrid assays of protein/protein interactions can be used (see e.g., Chien et al., Proc. Natl. Acad. Sci. USA, 88:9578, 1991; Fields et al., U.S. Pat. No. 5,283,173; Fields and Song, Nature, 340:245, 1989; Le Douarin et al., Nucleic Acids Research, 23:876, 1995; Vidal et al., Proc. Natl. Acad. Sci. USA, 93:10315-10320, 1996; and White, Proc. Natl. Acad. Sci. USA, 93:10001-10003, 1996). Generally, the two-hybrid hybrid methods involve in vivo reconstitution of two separable domains of a transcription factor. One fusion protein contains the Kin28 polypeptide fused to either a transactivator domain or DNA binding domain of a transcription factor (e.g., of Gal4). The other fusion protein contains a test polypeptide fused to either the DNA binding domain or a transactivator domain of a transcription factor. Once brought together in a single cell (e.g., a yeast cell or mammalian cell), one of the fusion proteins contains the transactivator domain and the other fusion protein contains the DNA binding domain. Therefore, binding of the Kin28 polypeptide to the test polypeptide (i.e., candidate antifungal agent) reconstitutes the transcription factor. Reconstitution of the transcription factor can be detected by detecting expression of a gene (i.e., a reporter gene) that is operably linked to a DNA sequence that is bound by the DNA binding domain of the transcription factor. Kits for practicing various two-hybrid methods are commercially available (e.g., from Clontech; Palo Alto, Calif.).

The methods described above can be used for high throughput screening of numerous test compounds to identify candidate antifungal (or anti-fungal) agents. Having identified a test compound as a candidate antifungal agent, the candidate antifungal agent can be further tested for inhibition of fungal growth in vitro or in vivo (e.g., using an animal, e.g., rodent, model system) if desired. Using other, known variations of such methods, one can test the ability of a nucleic acid (e.g., DNA or RNA) used as the test compound to bind to Kin28.

In vitro, further testing can be accomplished by means known to those in the art such as an enzyme inhibition assay or a whole-cell fungal growth inhibition assay. For example, an agar dilution assay identifies a substance that inhibits fungal growth. Microtiter plates are prepared with serial dilutions of the test compound, adding to the preparation a given amount of growth substrate, and providing a preparation of fungi. Inhibition of fungal growth is determined, for example, by observing changes in optical densities of the fungal cultures.

Inhibition of fungal growth is demonstrated, for example, by comparing (in the presence and absence of a test compound) the rate of growth or the absolute growth of fungal cells. Inhibition includes a reduction in the rate of growth or absolute growth by at least 20%. Particularly potent test compounds can further reduce the growth rate (e.g., by at least 25%, 30%, 40%, 50%, 75%, 80%, or 90%).

Animal (e.g., rodent such as murine) models of fungal infections are known to those of skill in the art, and such animal model systems are acceptable for screening antifungal agents as an indication of their therapeutic efficacy in human patients. In a typical in vivo assay, an animal is infected with a pathogenic strain of fungi, e.g., by inhalation of fungi, and conventional methods and criteria are used to diagnose the mammal as being afflicted with a fungal infection. The candidate antifungal agent then is administered to the mammal at a dosage of 1-100 mg/kg of body weight, and the mammal is monitored for signs of amelioration of disease. Alternatively, the test compound can be administered to the mammal prior to infecting the mammal with the fungi, and the ability of the treated mammal to resist infection is measured. Of course, the results obtained in the presence of the test compound should be compared with results in control animals, which are not treated with the test compound. Administration of candidate antifungal agents to the mammal can be carried out as described below, for example.

Antifungal agents can be identified with high throughput assays to detect inhibition of Kin28 activity, e.g., the ability to associate with a cyclin, e.g., Ccl1, the ability to promote RNA polymerase II-mediated transcription, and/or the ability to mediate serine/threonine kinase activity, e.g., the ability to phosphorylate the CTD of RNA polymerase II. For example, this inhibition can be caused by small molecules binding directly to the Kin28 polypeptide, e.g., the kinase domain of the Kin28 polypeptide, or by binding of small molecules to other essential polypeptides in a biochemical pathway in which Kin28 participates.

The invention also provides methods of identifying agents (such as compounds, other substances, or compositions) that affect, or selectively affect, (such as inhibit or otherwise modify) the activity of and/or expression of Kin28 polypeptides, by contacting Kin28 or the nucleotide sequence encoding the same with the agent and then measuring the activity of Kin28, e.g., kinase activity, and/or the expression thereof. In a related aspect, the invention features a method of identifying agents (such as compounds, other substances or compositions comprising same) that affect (such as inhibit or otherwise modify) the activity of and/or expression of KIN28 nucleic acids, by measuring the activity of and/or expression of KIN28 in the presence of the agent or after the addition of the agent in: (a) a cell line into which has been incorporated a recombinant construct including the nucleotide sequence of the KIN28 gene (e.g., SEQ ID NO: 1, SEQ ID NO: 2, SEQ ID NO: 3, SEQ ID NO: 4, or SEQ ID NO: 5) or an allelic variation thereof; or (b) a cell population or cell line that naturally selectively expresses KIN28, and then measuring the activity of KIN28 and/or the expression thereof.

Since the Aspergillus KIN28 gene described herein has been identified, it can be cloned into various host cells (e.g., fungi, E. coli, or yeast) for carrying out such assays in whole cells. Similarly, conventional in vitro assays of kinase activity can be used with the Kin28 polypeptides of the invention.

The invention also includes a method for identifying an antifungal agent where the method entails: (a) contacting a Kin28 polypeptide with a test compound; (b) detecting binding of the test compound to the polypeptide; and (c) determining whether a test compound that binds to the polypeptide inhibits growth of Aspergillus, relative to growth of fungi cultured in the absence of the test compound, as an indication that the test compound is an antifungal agent. If desired, the test compound can be immobilized on a substrate, and binding of the test compound to Kin28 is detected as immobilization of Kin28 on the immobilized test compound. Immobilization of Kin28 on the test compound can be detected in an immunoassay with an antibody that specifically binds to Kin28.

The binding of a test compound to a Kin28 polypeptide can be detected in a conventional two-hybrid system for detecting protein/protein interactions (e.g., in yeast or mammalian cells). A test compound found to bind to Kin28 can be further tested for antifungal activity in a conventional susceptibility assay. Generally, in such two-hybrid methods, (a) Kin28 is provided as a fusion protein that includes the polypeptide fused to (i) a transcription activation domain of a transcription factor or (ii) a DNA-binding domain of a transcription factor; (b) the test polypeptide is provided as a fusion protein that includes the test polypeptide fused to (i) a transcription activation domain of a transcription factor or (ii) a DNA-binding domain of a transcription factor; and (c) binding of the test polypeptide to the polypeptide is detected as reconstitution of a transcription factor. Reconstitution of the transcription factor can be detected, for example, by detecting transcription of a gene that is operably linked to a DNA sequence bound by the DNA-binding domain of the reconstituted transcription factor (See, for example, White, 1996, Proc. Natl. Acad. Sci., 93:10001-10003 and references cited therein and Vidal et al., 1996, Proc. Natl. Acad. Sci., 93:10315-10320).

In an alternative method, an isolated nucleic acid molecule encoding a Kin28 is used to identify a compound that decreases the expression of Kin28 in vivo (e.g., in a Aspergillus cell). Such compounds can be used as antifungal agents. To discover such compounds, cells that express Kin28 are cultured, exposed to a test compound (or a mixture of test compounds), and the level of Kin28 expression or activity is compared with the level of Kin28 expression or activity in cells that are otherwise identical but that have not been exposed to the test compound(s). Standard quantitative assays of gene expression and Kin28 activity, e.g., kinase activity, can be used.

To identify compounds that modulate expression of the KIN28 gene the test compound(s) can be added at varying concentrations to the culture medium of Aspergillus. Such test compounds can include small molecules (typically, non-protein, non-polysaccharide chemical entities), polypeptides, and nucleic acids. The expression of KIN28 is then measured, for example, by Northern blot PCR analysis or RNAse protection analyses using a nucleic acid molecule of the invention as a probe. The level of expression in the presence of the test molecule, compared with the level of expression in its absence, will indicate whether or not the test molecule alters the expression of KIN28. Because KIN28 is essential for survival, test compounds that inhibit the expression and/or function of KIN28 are expected inhibit growth of, or kill, the cells that express KIN28.

More generally, binding of a test compound to a Kin28 polypeptide can be detected either in vitro or in vivo. If desired, the above-described methods for identifying compounds that modulate the expression of Kin28 polypeptides of the invention can be combined with measuring the levels of Kin28 expressed in cells, e.g., by carrying out an assay of Kin28 activity, e.g., kinase activity, as described above or, for example, performing a Western blot analysis using antibodies that bind to Kin28 The antifungal agents identified by the methods of the invention can be used to inhibit a spectrum of pathogenic or non-pathogenic fungal strains, e.g., Aspergillus species.

Some specific embodiments of the present invention relate to assay methods for the identification of antifungal agents using assays for antifungal agents which may be carried out both in whole cell preparations and in ex vivo cell-free systems. In each instance, the assay target is the KIN28 nucleotide sequence and/or the Kin28 polypeptide. Test compounds which are found to inhibit the KIN28 nucleotide sequence and/or Kin28 polypeptide in any assay method of the present invention are thus identified as potential or candidate antifungal agents. It is expected that the assay methods of the present invention will be suitable for both small and large-scale screening of test compounds as well as in quantitative assays such as serial dilution studies where the target KIN28 nucleotide sequence or the Kin28 polypeptide are exposed to a range of test compound concentrations.

When the assay methods of the present invention are carried out as a whole-cell assay, the target KIN28 nucleotide sequence and/or the Kin28 polypeptide and the entire living fungal cell may be exposed to the test compound under conditions normally suitable for growth. Optimal conditions including essential nutrients, optimal temperatures and other parameters, depending upon the particular fungal strain and suitable conditions being used, are well known in the art. Inhibition of expression of the KIN28 nucleotide sequence and/or the activity of Kin28 may be determined in a number of ways including observing the cell culture's growth or lack thereof. Such observation may be made visually, by optical densitometric or other light absorption/scattering means, or by yet other suitable means, whether manual or automated.

In the above whole-cell assay, an observed lack of cell growth may be due to inhibition of the KIN28 nucleotide sequence and/or Kin28 or may be due to an entirely different effect of the test compound, and further evaluation may be required to establish the mechanism of action and to determine whether the test compound is a specific inhibitor of the target. Accordingly, and in one embodiment of the present invention, the method may be performed as a paired-cell assay in which each test compound is separately tested against two different types of fungal cells, the first fungal cells having a target with altered properties that make them more susceptible to inhibition compared with that of the second fungal cells.

One manner of achieving differential susceptibility is by using mutant strains expressing a modified target Kin28 polypeptide. A particularly useful strain is one having a temperature sensitive (“ts”) mutation as a result of which the target is more prone than the wild type target to loss of functionality at high temperatures (that is, temperatures higher than optimal, but still permitting growth in wild type cells). When grown at semi-permissive temperatures, the activity of a ts mutant target may be attenuated but sufficient for growth.

Alternatively or in conjunction with the above, differential susceptibility to target inhibitors may be obtained by using a second fungal cell which has altered properties that make it less susceptible to inhibition compared with that of wild type cells such as, for example, a fungal cell that has been genetically manipulated to cause overexpression of a target of the inhibitor. Such overexpression can be achieved by placing into a wild type cell a plasmid carrying the nucleotide sequence for the target. The techniques for generating temperature sensitive mutants, for preparing specific plasmids, and for transforming cell lines with such plasmids are well known in the art.

Alternatively or in conjunction with the above, the access of test compounds to a cell or an organism may be enhanced by mutating or deleting a gene or genes which encode a protein or proteins responsible for providing a permeability barrier for a cell or an organism.

The present invention also relates to a method for identifying antifungal agents utilizing fungal cell systems that are sensitive to perturbation to one or several transcriptional/translational components.

By way of example, the present invention relates to a method of constructing mutant fungal cells in which one or more of the transcriptional/translational components is present in an altered form or in a different amount compared with a corresponding wild-type cell. This method further involves examining a test compound for its ability to perturb transcription/translation by assessing the impact it has on the growth of the mutant and wild-type cells. Agents that perturb transcription/translation by acting on a particular component that participates in transcription/translation may cause a mutant fungal cell which has an altered form or amount of that component to grow differently from the corresponding wild-type cell, but do not affect the growth relative to the wild type cell of other mutant cells bearing alterations in other components participating in transcription/translation. This method thus provides not only a means to identify whether a test compound perturbs transcription/translation but also an indication of the site at which it exerts its effects. The transcriptional/translational component which is present in altered form or amount in a cell whose growth is affected by a test compound is likely to be the site of action of the agent.

By way of example, the present invention provides a method for identifying antifungal agents that interfere with steps in translational accuracy, such as maintaining a proper reading frame during translation and terminating translation at a stop codon. This method involves constructing mutant fungal cells in which a detectable reporter polypeptide can only be produced if the normal process of staying in one reading frame or of terminating translation at a stop codon has been disrupted. This method further involves contacting the mutant fungal cells with a test compound to examine whether it increases or decreases the production of the reporter polypeptide.

The present invention also provides a method of screening an agent for specific binding affinity with Kin28 (or a derivative, homolog, variant or fragment thereof) or the nucleotide sequence coding for same (including a derivative, homolog, variant or fragment thereof), the method comprising the steps of: a) providing a test compound; b) combining Kin28 (or the derivative, homolog, variant or fragment thereof) or the nucleotide sequence coding for same (or the derivative, homolog, variant or fragment thereof) with the test compound for a time sufficient to allow binding under suitable conditions; such binding or interaction being associated with a second component capable of providing a detectable signal in response to the binding or interaction of the Kin28 polypeptide or the nucleotide sequence encoding same with the agent; and c) determining whether the agent binds to or otherwise interacts with and activates or inhibits an activity of Kin28 (or the derivative, homolog, variant or fragment thereof) or the expression of the nucleotide sequence coding for same (or the derivative, homolog, variant or fragment thereof) by detecting the presence or absence of a signal generated from the binding and/or interaction of the agent with Kin28 (or the derivative, homolog, variant or fragment thereof) or the nucleotide sequence coding for same (or the derivative, homolog, variant or fragment thereof).

In other embodiments, the cell system is an extract of a fungal cell that is grown under defined conditions, and the method involves measuring transcription or translation in vitro. Such defined conditions are selected so that transcription or translation of the reporter is increased or decreased by the addition of a transcription inhibitor or a translation inhibitor to the cell extract.

One such method for identifying antifungal agents relies upon a transcription-responsive gene product. This method involves constructing a fungal cell in which the production of a reporter molecule, measured as a percentage of over-all transcription, increases or decreases under conditions in which overall fungal cell transcription is reduced. Specifically, the reporter molecule is encoded by a nucleic acid transcriptionally linked to a sequence constructed and arranged to cause a relative increase or decrease in the production of the reporter molecule when overall transcription is reduced. Typically, the overall transcription is measured by the expression of a second indicator gene whose expression, when measured as a percentage of overall transcription, remains constant when the overall transcription is reduced. The method further involves contacting the fungal cell with a test compound, and determining whether the test compound increases or decreases the production of the first reporter molecule in the fungal cell.

In one embodiment, the reporter molecule is itself the transcription-responsive gene product whose production increases or decreases when overall transcription is reduced. In another embodiment, the reporter is a different molecule whose production is linked to that of the transcription-responsive gene product. Such linkage between the reporter and the transcription-responsive gene product can be achieved in several ways. A gene sequence encoding the reporter may, for example, be fused to part or all of the gene encoding the transcription-responsive gene product and/or to part or all of the genetic elements which control the production of the gene product. Alternatively, the transcription-responsive gene product may stimulate transcription of the gene encoding the reporter, either directly or indirectly.

Alternatively, the method for identifying antifungal agents relies upon a translation-responsive gene product. This method involves constructing a fungal cell in which the production of a reporter molecule, measured as a percentage of over-all translation, increases or decreases under conditions in which overall fungal cell translation is reduced. Specifically, the reporter molecule is encoded by nucleic acid either translationally linked or transcriptionally linked to a sequence constructed and arranged to cause a relative increase or decrease in the production of the reporter molecule when overall translation is reduced. Typically, the overall translation is measured by the expression of a second indicator gene whose expression, when measured as a percentage of overall translation, remains constant when the overall translation is reduced. The method further involves contacting the fungal cell with a test compound, and determining whether the agent increases or decreases the production of the first reporter molecule in the fungal cell.

In one embodiment, the reporter molecule is itself the translation-responsive gene product whose production increases or decreases when overall translation is reduced. In another embodiment, the reporter is a different molecule whose production is linked to that of the translation-responsive gene product. Such linkage between the reporter and the translation-responsive gene product can be achieved in several ways. A gene sequence encoding the reporter may, for example, be fused to part or all of the gene encoding the translation-responsive gene product and/or to part or all of the genetic elements which control the production of the gene product. Alternatively, the translation-responsive gene product may stimulate translation of the gene encoding the reporter, either directly or indirectly.

Generally, a wide variety of reporters may be used, with typical reporters providing conveniently detectable signals (e.g., by spectroscopy). By way of example, a reporter gene may encode an enzyme which catalyses a reaction which alters light absorption properties.

Examples of reporter molecules include but are not limited to e-galactosidase, invertase, green fluorescent protein, luciferase, chloramphenicol, acetyltransferase, beta-glucuronidase, exo-glucanase and glucoamylase. Alternatively, radiolabeled or fluorescent tag-labeled nucleotides can be incorporated into nascent transcripts which are then identified when bound to oligonucleotide probes. For example, the production of the reporter molecule can be measured by the enzymatic activity of the reporter gene product, such as e-galactosidase.

In another embodiment of the present invention, a selection of hybridization probes corresponding to a predetermined population of genes of the selected fungal organism may be used to specifically detect changes in gene transcription which result from exposing the selected organism or cells thereof to a test compound. In this embodiment, one or more cells derived from the organism is exposed to the test compound in vivo or ex vivo under conditions wherein the agent effects a change in gene transcription in the cell to maintain homeostasis. Thereafter, the gene transcripts, primarily mRNA, of the cell or cells are isolated by conventional means. The isolated transcripts or cDNAs complementary thereto are then contacted with an ordered matrix of hybridization probes, each probe being specific for a different one of the transcripts, under conditions where each of the transcripts hybridizes with a corresponding one of the probes to form hybridization pairs. The ordered matrix of probes provides, in aggregate, complements for an ensemble of genes of the organism sufficient to model the transcriptional responsiveness of the organism to a test compound. The probes are generally immobilized and arrayed onto a solid substrate such as a microtiter plate. Specific hybridization may be effected, for example, by washing the hybridized matrix with excess non-specific oligonucleotides. A hybridization signal is then detected at each hybridization pair to obtain a transcription signal profile. A wide variety of hybridization signals may be used. In one embodiment, the cells are pre-labeled with radionucleotides such that the gene transcripts provide a radioactive signal that can be detected in the hybridization pairs. The transcription signal profile of the agent-treated cells is then compared with a transcription signal profile of negative control cells to obtain a specific transcription response profile to the test compound.

A variety of protocols for detecting and measuring the expression of Kin28, using either polyclonal or monoclonal antibodies specific for the protein, are known in the art. Examples include enzyme-linked immunosorbent assay (ELISA), radioimmunoassay (RIA) and fluorescent activated cell sorting (FACS). A two-site, monoclonal-based immunoassay utilizing monoclonal antibodies reactive to two non-interfering epitopes on Kin28 polypeptides is suitable; alternatively, a competitive binding assay may be employed. These and other assays are described, among other places, in Hampton, R et al. (1990, Serological Methods, A Laboratory Manual, APS Press, St Paul, Minn.) and Maddox, Del. et al. (1983, J. Exp. Med. 158:121).

In an embodiment of the present invention, Kin28 or a variant, homolog, fragment or derivative thereof and/or a cell line that expresses Kin28 or variant, homolog, fragment or derivative thereof may be used to screen for antibodies, peptides, or other agents, such as organic or inorganic molecules, that act as modulators of Kin28 activity, thereby identifying a therapeutic agent capable of modulating the activity of Kin28 For example, antibodies that specifically bind to a Kin28 polypeptide and are capable of neutralizing the activity of Kin28 may be used to inhibit Kin28 activity. Alternatively, screening of peptide libraries or organic libraries made by combinatorial chemistry with a recombinantly expressed Kin28 polypeptide or a variant, homolog, fragment or derivative thereof or cell lines expressing Kin28 or a variant, homolog, fragment or derivative thereof may be useful for identification of therapeutic agents that function by modulating Kin28 activity. Synthetic compounds, natural products, and other sources of potentially biologically active materials can be screened using any of a number of known screening methods. For example, nucleotide sequences encoding the N-terminal region of Kin28 can be expressed in a cell line and used for screening of allosteric modulators, either agonists or antagonists, of Kin28 activity.

Accordingly, the present invention provides a method for screening a plurality of agents for specific binding affinity with Kin28, or a portion, variant, homolog, fragment or derivative thereof, by providing a plurality of agents; combining Kin28 or a portion, variant, homolog, fragment or derivative thereof with each of a plurality of agents for a time sufficient to allow binding under suitable conditions; and detecting binding of Kin28, or portion, variant, homolog, fragment or derivative thereof, to each of the plurality of agents, thereby identifying the agent or agents which specifically bind Kin28 In such an assay, the plurality of agents may be produced by combinatorial chemistry techniques known to those of skill in the art.

Another technique for screening provides for high throughput screening of agents having suitable binding affinity to Kin28 polypeptides and is based upon the method described in detail in WO 84/03564. In summary, large numbers of different small peptide test compounds are synthesized on a solid substrate, such as plastic pins or some other surface. The peptide test compounds are reacted with Kin28 fragments and washed. A bound Kin28 polypeptide is then detected, such as by appropriately adapting methods well known in the art. A purified Kin28 polypeptide can also be coated directly onto plates for use in the aforementioned drug screening techniques. Alternatively, non-neutralizing antibodies can be used to capture the peptide and immobilize it on a solid support.

Typically, in an antifungal discovery process, potential new antifungal agents are tested for their ability to inhibit the in vitro activity of the purified expression product of the present invention in a biochemical assay. Agents with inhibitory activity can then progress to an in vitro antifungal activity screening using a standard MIC (Minimum Inhibitory Concentration) test (based on the M27-A NCCLS approved method). Antifungal agents identified at this point are then tested for antifungal efficacy in vivo, such as by using rodent systemic candidiasis/aspergillosis models. Efficacy is measured by measuring the agent's ability to increase the host animal's survival rate against systemic infection, and/or reduce the fungal burden in infected tissues, compared to control animals receiving no administered agent (which can be by oral or intravenous routes).

Medicinal Chemistry

Once a compound has been identified as an antifungal agent, principles of standard medicinal chemistry can be used to produce derivatives of the compound. Derivatives can be screened for improved pharmacological properties, for example, efficacy, pharmacokinetics, mammalian toxicity, stability, solubility, and clearance. The moieties that are responsible for the compound's activity can be revealed by examining its structure-activity relationships (SAR). Specifically, a person of ordinary skill in the art of chemistry can modify a moiety of the compound to study the effects of the modification on the potency of the compound and thereby produce derivatives of the compound having increased potency (See, e.g., Nagarajan et al., Antibiot. 41:430-438). For example, chemical modifications such as N-acylation, esterification, hydroxylation, alkylation, amination, amidation, oxidation, or reduction can be made. Such chemical modifications can be made according to conventional methods (See, e.g., Wade, Organic Chemistry, Prentice-Hall, Inc., New Jersey, 1987). In addition, structural information can be used to design and optimize derivatives of the inhibitor by using molecular modeling software and conventional methods. Molecular modeling software is commercially available (e.g., from Tripos Inc., Molecular Simulations, Inc., and MDL Information Systems, Inc).

Pharmaceutical Formulations

The present invention also provides pharmaceutical composition or formulation for treating an individual in need of such treatment of a disease caused by Aspergillus (or that can be treated by inhibiting Kin28 activity); the treatment method entails administering a therapeutically effective amount of an agent that affects (such as inhibits) the activity and a pharmaceutically acceptable carrier, diluent, excipient, or adjuvant.

The pharmaceutical compositions described herein typically include any one or more of a pharmaceutically acceptable diluent, carrier, excipient, or adjuvant. The pharmaceutical carrier, excipient, or diluent can be selected with regard to the intended route of administration and standard pharmaceutical practice. The pharmaceutical compositions can include as (or in addition to) the carrier, excipient, or diluent, any suitable binder(s), lubricant(s), suspending agent(s), coating agent(s), or solubilizing agent(s).

The pharmaceutical formulations can contain antifungal agents that inhibit the growth of, or kill, pathogenic fungal strains (e.g., pathogenic Aspergillus strains), e.g., antifungal agents identified by the methods described herein. Such pharmaceutical formulations can be used in methods of treating fungal infections in organisms, e.g., in mammals such as humans and domesticated mammals (e.g., cows, pigs, dogs, and cats), in birds such as poultry, and in plants. The method entails administering to the organism a therapeutically effective amount of the pharmaceutical formulation, e.g., an amount sufficient to ameliorate signs and/or symptoms of the fungal infection. The efficacy of such antifungal agents in humans can be estimated in an animal model system well known to those of skill in the art (e.g., mouse systems of fungal infections).

Treatment includes administering a pharmaceutically effective amount of a composition containing an antifungal agent to a subject in need of such treatment, thereby inhibiting or reducing fungal growth in the subject. Such a composition typically contains from about 0.1 to 90% by weight (such as 1 to 20% or 1 to 10%) of an antifungal agent of the invention in a pharmaceutically acceptable carrier.

Solid formulations of the compositions for oral administration can contain suitable carriers or excipients, such as corn starch, gelatin, lactose, acacia, sucrose, microcrystalline cellulose, kaolin, mannitol, dicalcium phosphate, calcium carbonate, sodium chloride, or alginic acid. Disintegrators that can be used include, without limitation, micro-crystalline cellulose, corn starch, sodium starch glycolate and alginic acid. Tablet binders that can be used include acacia, methylcellulose, sodium carboxymethylcellulose, polyvinylpyrrolidone (Povidone®), hydroxypropyl methylcellulose, sucrose, starch, and ethylcellulose. Lubricants that can be used include magnesium stearates, stearic acid, silicone fluid, talc, waxes, oils, and colloidal silica.

Liquid formulations of the compositions for oral administration prepared in water or other aqueous vehicles can contain various suspending agents such as methylcellulose, alginates, tragacanth, pectin, kelgin, carageenan, acacia, polyvinylpyrrolidone, and polyvinyl alcohol. The liquid formulations can also include solutions, emulsions, syrups and elixirs containing, together with the active compound(s), wetting agents, sweeteners, and coloring and flavoring agents. Various liquid and powder formulations can be prepared by conventional methods for inhalation into the lungs of the mammal to be treated.

Injectable formulations of the compositions can contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol, and the like). For intravenous injections, water soluble versions of the compounds can be administered by the drip method, whereby a pharmaceutical formulation containing the antifungal agent and a physiologically acceptable excipient is infused. Physiologically acceptable excipients can include, for example, 5% dextrose, 0.9% saline, Ringer's solution, or other suitable excipients. For intramuscular preparations, a sterile formulation of a suitable soluble salt form of the compounds can be dissolved and administered in a pharmaceutical excipient such as Water-for-Injection, 0.9% saline, or 5% glucose solution. A suitable insoluble form of the compound can be prepared and administered as a suspension in an aqueous base or a pharmaceutically acceptable oil base, such as an ester of a long chain fatty acid, (e.g., ethyl oleate).

A topical semi-solid ointment formulation typically contains a concentration of the active ingredient from about 1 to 20%, e.g., 5 to 10% in a carrier such as a pharmaceutical cream base. Various formulations for topical use include drops, tinctures, lotions, creams, solutions, and ointments containing the active ingredient and various supports and vehicles.

The optimal percentage of the antifungal agent in each pharmaceutical formulation varies according to the formulation itself and the therapeutic effect desired in the specific pathologies and correlated therapeutic regimens. Appropriate dosages of the antifungal agents can be determined by those of ordinary skill in the art of medicine by monitoring the mammal for signs of disease amelioration or inhibition, and increasing or decreasing the dosage and/or frequency of treatment as desired. The optimal amount of the antifungal agent used for treatment of conditions caused by or contributed to by fungal infection depends upon the manner of administration, the age and the body weight of the subject, and the condition of the subject to be treated. Generally, the antifungal compound is administered at a dosage of 1 to 100 mg/kg body weight, and typically at a dosage of 1 to 10 mg/kg body weight.

Methods of Treatment and Methods of Detection

The invention also includes (i) a method of treating a mycosal and/or fungal infection in a target (which target can be a living organism, such as a mammal, such as a human, or an inanimate target, such as a textile piece, paper, plastic etc.), which method entails delivering (such as administering or exposing) an effective amount of an agent capable of modulating the expression pattern of the nucleotide sequence of the present invention or the activity of the expression product thereof; and (ii) a method of treating a mycosal and/or fungal infection in a target (which target can be a living organism, such as a plant or a mammal, such as a human, or an inanimate target, such as a textile piece, paper, plastic, etc.), which method entails delivering (such as administering or exposing) an effective amount of an agent identified by an assay according to the present invention. As used herein, the terms “treating,” “treat,” or “treatment” include, inter alia, preventative (e.g., prophylactic), palliative, and curative treatment of fungal infections.

The invention also features a method for inducing an immunological response in an individual, particularly a mammal, which entails inoculating the individual with one or more of the Kin28 genes or polypeptides described herein, and generally in an amount adequate to produce an antibody and/or T cell immune response to protect the individual from mycoses, fungal infection, or infestations. In another aspect, the present invention relates to a method of inducing an immunological response in an individual which entails delivering to the individual a vector that includes a Kin28 gene described herein or a variant, homolog, fragment, or derivative thereof in vivo to induce an immunological response, such as to produce antibody and/or a T-cell immune response to protect the individual from disease, whether that disease is already established within the individual or not.

Various affinity reagents that are permeable to microbial membranes (i.e., antibodies and antibody fragments) are useful in practicing the methods of the invention. For example polyclonal and monoclonal antibodies that specifically bind to the Kin28 polypeptide can facilitate detection of Aspergillus Kin28 in various fungal strains (or extracts thereof). These antibodies also are useful for detecting binding of a test compound to Kin28 (e.g., using the assays described herein). In addition, monoclonal antibodies that specifically bind to Kin28 can themselves be used as antifungal agents.

In another aspect, the invention features a method for detecting a Kin28 polypeptide in a sample. This method includes: obtaining a sample suspected of containing a Kin28 polypeptide; contacting the sample with an antibody that specifically binds to a Kin28 polypeptide under conditions that allow the formation of complexes of the antibody and the Kin28 polypeptide; and detecting the complexes, if any, as an indication of the presence of a Kin28 polypeptide in the sample.

In all of the foregoing methods, homologs, orthologs, or variants of the KIN28 genes and polypeptides described herein can be substituted. While “homologs” are structurally similar genes contained within a species, “orthologs” are functionally equivalent genes from other species (within or outside of a given genus, e.g., from E. coli). The terms “variant,” “homolog,” or “fragment” in relation to the amino acid sequence of the Kin28 of the invention include any substitution, variation, modification, replacement, deletion, or addition of one or more amino acids from or to the sequence providing the resultant Kin28 polypeptide.

### Other Embodiments

It is to be understood that, while the invention has been described in conjunction with the detailed description thereof, the foregoing description is intended to illustrate and not limit the scope of the invention, which is defined by the scope of the appended claims. Other aspects, advantages, and modifications are within the scope of the following claims. For example, other known assays to detect interactions of test compounds with proteins, or to detect inhibition of fungal growth also can be used with the KIN28 genes. The invention also includes methods of making a pharmaceutical composition for use in inhibiting Aspergillus. Specifically, the methods include formulating a pharmaceutically acceptable excipient with an antifungal agent, such as those described herein.

What is claimed is:

1. An isolated nucleic acid molecule selected from the group consisting of:
(a) a nucleic acid molecule that encodes a polypeptide comprising the amino acid sequence of SEQ ID NO: 6 or SEQ ID NO: 7;
(b) a nucleic acid molecule that encodes a polypeptide comprising at least 20 contiguous amino acids of SEQ ID NO: 6 or SEQ ID NO: 7; and
(c) a nucleic acid molecule that encodes a variant of a polypeptide comprising the amino acid sequence of SEQ ID NO: 6 or SEQ ID NO: 7, wherein the nucleic acid molecule hybridizes under stringent conditions to a nucleic acid molecule consisting of the nucleotide sequence of SEQ ID NO: 1, SEQ ID NO: 2, SEQ ID NO: 3, SEQ ID NO: 4, or SEQ ID NO: 5, or a complement of SEQ ID NO: 1, SEQ ID NO: 2, SEQ ID NO: 3, SEQ ID NO: 4, or SEQ IL) NO: 5.

2. An isolated nucleic acid molecule selected from the group consisting of:
(a) a nucleic acid molecule comprising the nucleotide sequence of SEQ ID NO: 1, SEQ ID NO: 2, SEQ ID NO: 3, SEQ ID NO: 4, or SEQ ID NO: 5;
(b) a nucleic acid molecule comprising the nucleotide sequence of SEQ ID NO: 1, SEQ ID NO: 2, SEQ ID NO: 3, SEQ ID NO: 4, or SEQ ID NO: 5, wherein the thymines are replaced with uracils;
(c) a nucleic acid molecule that is complementary to (a) or (b); and
(d) fragments of (a), (b), or (c) that contain at least 50 contiguous nucleotides of SEQ ID NO: 1, SEQ ID NO: 2, SEQ ID NO: 3, SEQ ID NO: 4, or SEQ ID NO: 5 or a complement of SEQ ID NO: 1, SEQ ID NO: 2, SEQ ID NO: 3, SEQ ID NO: 4, or SEQ ID NO: 5.

3. An isolated nucleic acid molecule selected from the group consisting of:
(a) a nucleic acid molecule comprising a nucleotide sequence at least about 85% identical to the nucleotide sequence of SEQ ID NO: 1, SEQ ID NO: 2, SEQ ID NO: 3, SEQ ID NO: 4, or SEQ ID NO: 5 or a complement thereof, wherein the percent identity is calculated using the GAP program in the GCG software package, using a gap weight of 5.000 and a length weight of 0.100; and
(b) a nucleic acid molecule comprising a nucleotide sequence that hybridizes to a nucleic acid molecule consisting of the nucleotide sequence of SEQ ID NO: 1, SEQ ID NO: 2, SEQ ID NO: 3, SEQ ID NO: 4, or SEQ ID NO: 5 under stringent conditions, or a complement thereof.

4. The nucleic acid molecule of claim 1, further comprising a vector nucleic acid sequence.

5. The nucleic acid molecule of claim 1, further comprising a nucleic acid sequence encoding a heterologous polypeptide.

6. A host cell comprising the nucleic acid molecule of claim 1.

7. The host cell of claim 6, wherein the host cell is a mammalian cell.

8. The host cell of claim 6, wherein the host cell is a non-mammalian cell.

9. A method for producing a polypeptide, the method comprising culturing the host cell of claim 6 under conditions in which the nucleic acid molecule is expressed.

10. An isolated polypeptide selected from the group consisting of:
(a) a polypeptide comprising the amino acid sequence of SEQ ID NO: 6 or SEQ ID NO: 7;
(b) a polypeptide comprising a sequence of at least 20 contiguous amino acids of SEQ ID NO: 6 or SEQ ID NO: 7;
(c) a variant of a polypeptide comprising the amino acid sequence of SEQ ID NO: 6, or SEQ ID NO: 7, wherein the polypeptide is encoded by a nucleic acid molecule that hybridizes under stringent conditions to the complement of a nucleic acid molecule consisting of the nucleotide sequence of SEQ ID NO: 1, SEQ ID NO: 2, SEQ ID NO: 3, SEQ ID NO: 4, or SEQ ID NO: 5;
(d) a polypeptide encoded by a nucleic acid molecule comprising a nucleotide sequence that is at least 85% identical to SEQ ID NO: 1, SEQ ID NO: 2, SEQ ID NO: 3, SEQ ID NO: 4, or SEQ ID NO: 5, wherein the percent identity is calculated using the GAP program in the GCG software package, using a gap weight of 5.000 and a length weight of 0.100; and
(e) a polypeptide comprising SEQ ID NO: 6 with up to 10 conservative amino acid substitutions, or SEQ ID NO: 7 with up to 10 conservative amino acid substitutions, wherein the polypeptide mediates serine/threonine protein kinase activity.

11. An antibody that selectively binds to the polypeptide of claim 10.

12. A host cell comprising a nucleic acid molecule that encodes the polypeptide of claim 10.

13. A method for producing a polypeptide, the method comprising culturing the host cell of claim 12 under conditions in which the nucleic acid molecule is expressed.

14. A method for identifying a candidate antifungal agent, the method comprising:
(a) obtaining a first cell and a second cell, the first and second cells being capable of expressing the KIN28 nucleic acid molecule of claim 1;
(b) contacting the first cell with a test compound;
(c) determining the level of expression of KIN28 in the first and second cells; and
(d) comparing the level of expression in the first cell with the second cell; wherein expression of KIN28 in the first cell less than expression of KIN28 in the second cell indicates that the test compound is a candidate antifungal agent.

15. The method of claim 14, wherein the level of expression is measured by measuring the amount of KIN28 mRNA in the cell.

16. The method of claim 14, wherein the level of expression is measured by measuring the amount of protein encoded by Kin28.

17. The method of claim 14, wherein the test compound is a compound selected from the group consisting of: a polypeptide, a ribonucleic acid, a small organic molecule, a small inorganic molecule, a deoxyribonucleic acid, an antisense oligonucleotide, a peptidomimetic, a polysaccharide, and a ribozyme.

18. A method for identifying an agent that can affect KIN28 nucleic acid molecule expression, the method comprising:
contacting an agent with the nucleic acid molecule of claim 1, and measuring the expression of the nucleic acid molecule, where a difference between a) expression in the absence of the agent and b) expression in the presence of the agent indicates that the agent can affect KIN28 expression.

19. A method for identifying an agent that can affect Kin28 polypeptide activity, the method comprising:
contacting an agent with the polypeptide of claim 10; and
measuring the activity of the polypeptide; where a difference between a) activity in the absence of the agent and b) activity in the presence of the agent indicates that the agent can affect Kin28 polypeptide activity.

20. A method for identifying a candidate compound for treating a fungal infection, the method comprising:
(a) measuring the activity of a KIN28 gene comprising the sequence of SEQ ID NO: 1 or SEQ ID NO: 3 in a cell in the presence of a test compound; and
(b) comparing the activity measured in step (a) to a level of activity of the KIN28 gene in a cell in the absence of the test compound; wherein a level of activity of the KIN28 gene measured in the presence of the test compound lower than the level of activity of the KIN28 gene measured in the absence of the test compound indicates that the test compound is a candidate compound for treating a fungal infection.

21. A method for identifying an antifungal agent for the treatment of a fungal infection, the method comprising:
(a) obtaining a first sample of cells and a second sample of cells, the first and second samples of cells being capable of expressing the nucleic acid molecule of claim 1 in the presence of a test compound;
(b) contacting the first sample of cells with a test compound; and
(c) comparing the growth of the first sample of cells with the growth of the second sample of cells; wherein growth of the first sample of cells slower than the growth of the second sample of cells indicates the test compound is an antifungal agent.

22. The method of claim 21, wherein the first and second samples of cells comprise fungal cells of the genus Aspergillus.

23. The method of claim 22, wherein the fungal cells are Aspergillus nidulans cells.

24. The method of claim 22, wherein the fungal cells are Aspergillus fumigatus cells.

25. A method for identifying a candidate compound for treating a fungal infection, the method comprising:
(a) contacting a Kin28 polypeptide of claim 10 with a test compound; and
(b) detecting binding of the test compound to the polypeptide, wherein a compound that binds to the Kin28 polypeptide indicates that the test compound is a candidate compound for treating a fungal infection.

26. The method of claim 25, further comprising:
(c) determining whether the candidate compound that binds to the Kin28 polypeptide inhibits growth of fungi, relative to growth of fungi grown in the absence of the test compound, wherein inhibition of growth indicates that the candidate compound is an antifungal agent.

27. The method of 25, wherein the test compound is immobilized on a substrate, and binding of the test compound to the Kin28 polypeptide is detected as immobilization of the Kin28 polypeptide on the immobilized test compound.

28. The method of claim 25, wherein immobilization of the Kin28 polypeptide on the test compound is detected in an immunoassay with an antibody that specifically binds to the Kin28 polypeptide.

29. The method of claim 25, wherein the test compound is selected from the group consisting of: a polypeptide, a ribonucleic acid, a small organic molecule, a small inorganic molecule, a deoxyribonucleic acid, an antisense oligonucleotide, a peptidomimetic, a polysaccharide, and a ribozyme.

30. The method of claim 25, wherein the Kin28 polypeptide is provided as a first fusion protein comprising a Kin28 polypeptide fused to (i) a transcription activation domain of a transcription factor or (ii) a DNA-binding domain of a transcription factor; and the test compound is a polypeptide that is provided as a second fusion protein comprising the test compound fused to (i) a transcription activation domain of a transcription factor or (ii) a DNA-binding domain of a transcription factor, to interact with the first fusion protein; and binding of the test compound to the Kin28 polypeptide is detected as reconstitution of a transcription factor.

31. A pharmaceutical formulation for the treatment of a fungal infection, the formulation comprising an antifungal agent identified by the method of claim 26 and a pharmaceutically acceptable excipient.

32. A method for treating an organism having a fungal infection, the method comprising administering to the organism a therapeutically effective amount of the pharmaceutical formulation of claim 31.

33. The method of claim 32, wherein the organism is a human.

34. A method of treating an antifungal infection in an organism, the method comprising administering to the organism a therapeutically effective amount of the antibody of claim 11.

35. The method of claim 34, wherein the antibody is a monoclonal antibody.

36. A pharmaceutical formulation for the treatment of a fungal infection in an organism, the formulation comprising a ribozyme of claim 29 and a pharmaceutically acceptable excipient.

37. A pharmaceutical formulation for the treatment of a fungal infection in an organism, the formulation comprising the antisense nucleic acid of claim 29 and a pharmaceutically acceptable excipient.

38. A method for identifying a candidate compound for treating a fungal infection, the method comprising:
(a) contacting the nucleic acid molecule of claim 1 with a test compound; and
(b) detecting binding of the test compound to the nucleic acid molecule, wherein a compound that binds to the nucleic acid molecule is a candidate compound for treating a fungal infection.

39. The method of claim 38, further comprising determining whether a candidate compound that binds to the nucleic acid molecule inhibits growth of fungi, relative to growth of fungi grown in the absence of the test compound, wherein inhibition of growth indicates that the candidate compound is an antifungal agent.

40. The method of claim 38, wherein the test compound is selected from the group consisting of: a polypeptide, a ribonucleic acid, a small organic molecule, a small inorganic molecule, a deoxyribonucleic acid, an antisense oligonucleotide, a peptomimetic, a polysaccharide, and a ribozyme.

41. The method of claim 38, wherein the nucleic acid molecule is derived from a non-pathogenic fungus.

42. The method of claim 38, wherein the nucleic acid molecule is derived from a pathogenic fungus.

43. The method of claim 38, wherein the test compound is immobilized on a substrate, and binding of the test compound to the nucleic acid molecule is detected as immobilization of the nucleic acid molecule on the immobilized test compound.
